(19) United States
(12) Patent Application Publication
(10) Pub. No.: US 2007/0153972 A1
(43) Pub. Date: Jul. 5, 2007

(54) X-RAY CT APPARATUS

(76) Inventors: Takashi Fujishige, Tokyo (JP); Yasuro Takiura, Tokyo (JP); Akihiko Nishide, Tokyo (JP)

Correspondence Address: Patrick W. Rasche;Armstrong Teasdale LLP, One Metropolitan Square, Suite 2600, St. Louis, MO, 63102, US

(21) Appl. No.: 11/561,433

(22) Filed: Nov. 20, 2006

(30) Foreign Application Priority Data
Nov. 22, 2005 (JP) 2005-336852

(51) Int. Cl. H05G 1/60; A61B 6/00; G01N 23/00; G21K 1/12

(52) U.S. Cl. 378/19

(57) ABSTRACT

The present invention aims to optimize image quality for a conventional scan (axial scan) or a cine scan or a helical scan of an X-ray CT apparatus by a data acquisition system having a limited number of channels. The optimum view numbers determined or defined by image quality to be determined depending upon the positions of the respective channels at image reconstruction are determined by a sampling theorem. Thus, the optimum view numbers, which depend upon the respective channel positions, are allocated. The data acquisition system performs data acquisition in accordance with the views to make it possible to obtain a tomographic image having the optimum image quality. Thus, the number of A/D converters of the data acquisition system and its performance can also be optimized.

### BACKGROUND OF THE INVENTION

The present invention relates to an X-ray CT (Computed Tomography) imaging method suitable for use in a medical X-ray CT apparatus or an industrial X-ray CT apparatus, and an X-ray CT apparatus, and to a method of acquiring data at a conventional scan (axial scan) or a cine scan, or a helical scan.

An X-ray CT apparatus has heretofore performed data acquisition of X-ray detector all channels for every view at predetermined time intervals and data acquisition of view numbers identical even to any channel at X-ray data acquisition per rotation as shown in FIG. 7 (refer to Japanese Unexamined Patent Publication No. 2004-313657).

FIG. 7 shows X-ray detector data or projection data of an X-ray detector corresponding to one row. The X-ray detector data or projection data are X-ray data acquired from a 360-degree direction over the circumference of a subject. Its data acquisition angle is called view direction. The horizontal axis of FIG. 7 indicates a channel direction of the X-ray detector, and the vertical axis thereof indicates data acquisition in the view direction, i.e., 360-degree direction of the X-ray detector.

It was common that upon the conventional data acquisition as shown in FIG. 7, the number of data acquisitions in the view direction of 360° per rotation (hereinafter called view number) was identical to any channel.

With multichanneling and multi-row configuring of the X-ray CT apparatus, however, the number of all channels of the X-ray detector including the number of channel and row directions increases and the number of A/D converters of a data acquisition system (DAS) also increases, in a multi-row X-ray detector type X-ray CT apparatus or an X-ray CT apparatus based on a two-dimensional X-ray area detector typified by a flat panel. There has also been a demand for increases in its performance and throughput. From the viewpoint that both packaging and cost setting are being directed to their difficulties, the increases in performance and throughput dependant on the product of the number of all channels and the number of views in the data acquisition system result in problems.

Therefore, an object of the present invention is to provide an X-ray CT apparatus that reduces the number of X-ray data acquisition views of a data acquisition system (DAS) of an X-ray CT apparatus having an X-ray detector corresponding to one row, or an X-ray CT apparatus having a multi-row X-ray detector or a two-dimensional area X-ray detector of a matrix structure typified by a flat panel X-ray detector, and implements optimization of required performance and throughput of the data acquisition system (DAS).

### SUMMARY OF THE INVENTION

The present invention provides an X-ray CT apparatus or an X-ray CT imaging method which implements a data acquisition system (DAS) that performs data acquisition by optimization of view numbers dependant on channel positions of an X-ray detector and the data acquisition system (DAS).

On an image reconstruction plane (CT or tomographic image plane), a tomographic image is image-reconstructed by convoluting a reconstruction function on pre-processed projection data and effecting a backprojection process corresponding to 360° (or 180°+X-ray detector fan angles) thereon.

Upon the backprojection process, data backprojection is made in the 360-degree direction (or X-ray detector fan angles) with a reconstruction center and a tomographic image center each corresponding to the center of rotation as the center as shown in FIG. 8. Therefore, the resolution in the circumferential direction, of each pixel located in an area placed on a peripheral portion distant from the tomographic image center, i.e., a radius long as viewed from the tomographic image center depends on the number of views. That is, if sufficient view numbers exist, then the resolution of each pixel at the peripheral portion is ensured. If not so, then the resolution thereof is degraded.

Even though the neighborhood of the tomographic image center is short in circumference and the number of views is not so provided, the resolution on tomographic image space can be ensured. Generally, assuming that the size of one pixel is expressed as P×P, the radius of the neighborhood of the tomographic image center is given as r₁, and the radius of the peripheral portion of the tomographic image is given as r₂, for example, the following are given,

necessary view number V1=2πr1/P because of the circumference 2πr1 of the radius r1,

necessary view number V2=2πr2/P because of the circumference 2πr2 of the radius r2,

r1=50 mm,

r2=250 mm, and

p=500 mm/500 pixels=1 mm/1 pixel,

V1 and V2 result in V1=2π·50/1=314 views and V2=2π·250/1=1570 views.

On X-ray detector data or projection data at this time, X-ray detector data or projection data D (view, i) placed in a position spaced a distance r₁ or r₂ from the reconstruction center position (tomographic image center) serve so as to image-reconstruct a pixel on the circumference spaced a radius r₁ or r₂ from the tomographic image center as shown in FIG. 8. Here, view is assumed to be a view number and i is assumed to be a channel number.

Therefore, if the number of views is increased as the peripheral portion approaches, in proportion to the distance from a channel position corresponding to the tomographic image center to each channel, the resolution on the tomographic image dependant on the number of views can be kept uniform.

In a first aspect, the present invention provides an X-ray CT apparatus comprising X-ray data acquisition means for acquiring X-ray projection data transmitted through a subject lying between an X-ray generator and an X-ray detector detecting X rays in opposition to the X-ray generator, while the X-ray generator and the X-ray detector are being rotated about the center of rotation lying therebetween, image reconstructing means for image-reconstructing the projection data acquired from the X-ray data acquisition means, image display means for displaying an image-reconstructed tomographic image, and imaging condition setting means for setting various imaging conditions for tomographic-image photography, wherein X-ray data acquisition means is provided which performs X-ray data acquisition based on a plurality of types of X-ray data acquisition view numbers per rotation.

In the X-ray CT apparatus according to the first aspect, the view numbers for X-ray data acquisition are suitably applied to their corresponding channels thereby to make it possible to optimize the view numbers for the respective channels without degrading image quality of a CT or tomographic image.

In a second aspect, the present invention provides an X-ray CT apparatus wherein in the X-ray CT apparatus according to the first aspect, X-ray data acquisition means is provided which performs X-ray data acquisition at a plurality of types of different X-ray data acquisition view numbers depending upon channel positions.

In the X-ray CT apparatus according to the second aspect, the view numbers for the X-ray data acquisition relates to pixel resolution of a tomographic image existing along the circumference of a circle placed in the center of the tomographic image for every channel position. Therefore, the view number can be optimized by allowing each pixel placed on the circumference thereof to depend on its corresponding image-reconstructed channel position.

In a third aspect, the present invention provides an X-ray CT apparatus wherein in the X-ray CT apparatus according to each of the first and second aspects, X-ray data acquisition means is provided which acquires X-ray data small in view number in channels located in the vicinity of the center of rotation and large in view number in channels at positions spaced away from an X-ray detector channel position passing through the center of rotation.

In the X-ray CT apparatus according to the third aspect, the number of views is reduced since the distance from the center of rotation decreases in the channels located in the neighborhood of the center of rotation, whereas since the distance from the center of rotation increases in the channels distant from the center of rotation, the number of views is made large.

In a fourth aspect, the present invention provides an X-ray CT apparatus wherein in the X-ray CT apparatus according to the first or third aspect, X-ray data acquisition means is provided which performs X-ray data acquisition at a plurality of types of different X-ray data acquisition view numbers depending upon distances from an X-ray detector channel position passing through the center of rotation to respective channel positions.

In the X-ray CT apparatus according to the fourth aspect, the view numbers for the X-ray data acquisition depends on pixel resolution of a tomographic image existing along the circumference of a circle placed in the center of the tomographic image for every channel position. This circumference corresponds to the circumference of a circle in which the distance from the X-ray detector channel position passing through the center of the tomographic image to each channel position is defined as its radius. The respective X-ray detector channels image-reconstruct the pixels on the circumference. Therefore, the view numbers can be optimized by determining the X-ray data acquisition view numbers depending upon the distances from the X-ray detector channel position passing through the center of rotation to the respective channel positions.

In a fifth aspect, the present invention provides an X-ray CT apparatus wherein in the X-ray CT apparatus according to each of the first to fourth aspects, X-ray data acquisition means is provided which performs X-ray data acquisition at plural types of view numbers, based on X-ray data acquisition view numbers proportional to distances from an X-ray detector channel position passing through the center of rotation to respective channel positions, or about the X-ray data acquisition view numbers.

In the X-ray CT apparatus according to the fifth aspect, the view numbers for the X-ray data acquisition image-reconstruct a tomographic image placed on the circumference of a circle with the center of the tomographic image as the center for every channel position. Each of lengths obtained by dividing this circumference by the number of views depends upon the resolution of a pixel at each position of the tomographic image. Therefore, the view numbers can be optimized by determining the X-ray data acquisition view numbers in proportion to the distances from the X-ray detector channel position passing through the center of rotation to the respective channel positions.

In a sixth aspect, the present invention provides an X-ray CT apparatus wherein in the X-ray CT apparatus according to each of the first to fifth aspects, X-ray data acquisition means is provided which performs X-ray data acquisition at view numbers different for every channel, depending upon each reconstruction function.

In the X-ray CT apparatus according to the sixth aspect, the resolution of an XY plane corresponding to a tomographic image plane varies depending upon each reconstruction function. Therefore, the view numbers set for every channel position can be optimized by varying in accordance with the resolution of the XY plane that varies for every reconstruction function.

In a seventh aspect, the present invention provides an X-ray CT apparatus wherein in the X-ray CT apparatus according to each of the first to sixth aspects, X-ray data acquisition means is provided which performs X-ray data acquisition at view numbers different for every channel, depending upon the size of each imaging view field.

In the X-ray CT apparatus according to the seventh aspect, the required number of channels varies depending upon the size of each imaging view field. Therefore, the view numbers set for every channel position can be optimized by varying in accordance with the size of each imaging view field.

In an eighth aspect, the present invention provides an X-ray CT apparatus wherein in the X-ray CT apparatus according to each of the first to seventh aspects, X-ray data acquisition means is provided which performs X-ray data acquisition at view numbers different for every channel, depending upon z-direction coordinate positions.

In the X-ray CT apparatus according to the eighth aspect, the optimum imaging view fields corresponding to respective regions of a subject vary depending on the respective coordinate positions in the z direction. Therefore, the view number set for every channel position can be optimized by varying in match with the size of the imaging view field at each z-direction position corresponding to the size of a section of the subject.

In a ninth aspect, the present invention provides an X-ray CT apparatus wherein in the X-ray CT apparatus according to each of the first to eighth aspects, X-ray data acquisition means is provided which acquires X-ray data by a multi-row X-ray detector.

In the X-ray CT apparatus according to the ninth aspect, the multi-row X-ray detector cal also optimize X-ray data acquisition view numbers for every channel position.

In a tenth aspect, the present invention provides an X-ray CT apparatus wherein in the X-ray CT apparatus according to each of the first to eighth aspects, X-ray data acquisition means is provided which acquires X-ray data by a two-dimensional X-ray area detector of a matrix structure typified by a flat panel X-ray detector.

In the X-ray CT apparatus according to the tenth aspect, the two-dimensional X-ray area detector of matrix structure typified by the flat panel X-ray detector can also optimize X-ray data acquisition view numbers for every channel position.

In an eleventh aspect, the present invention provides an X-ray CT apparatus wherein in the X-ray CT apparatus according to each of the ninth and tenth aspects, X-ray data acquisition means is provided which performs data acquisition at X-ray data acquisition view numbers different for every channel independently for every row.

In the X-ray CT apparatus according to the eleventh aspect, when the optimum imaging view fields corresponding to the respective regions of the subject are varied according to the coordinate positions in the respective z directions, the X-ray data acquisition is performed at view numbers different for every channel position at the time of execution of one rotation or plural rotations for every z-direction coordinate position upon a conventional scan (axial scan) or a cine scan. Upon a helical scan or a variable pitch helical scan, the X-ray data acquisition view numbers can be optimized by varying at view numbers different for every channel position corresponding to each of imaging view-field sizes at z-direction positions, depending upon to which z-direction coordinate positions respective X-ray detector rows correspond.

According to the X-ray CT apparatus or the X-ray CT image reconstructing method, as the effects of the present invention, there can be provided an X-ray CT apparatus which reduces the number of X-ray data acquisition views in a data acquisition system (DAS) of an X-ray CT apparatus having a one-row type X-ray detector or an X-ray CT apparatus having a two-dimensional area X-ray detector of a matrix structure, which is typified by a multi-row X-ray detector or a flat panel X-ray detector and which implements optimization of required performance and throughput capacity of a data acquisition system (DAS).

### BRIEF DESCRIPTION OF THE DRAWINGS

FIG. 1 is a block diagram showing an X-ray CT apparatus according to a first embodiment of the present invention.

FIG. 2 is a diagram for describing rotation of an X-ray generator (X-ray tube) and a multi-row X-ray detector.

FIG. 3 is a flow chart showing an image reconstructing operation for correcting the number of views in the X-ray CT apparatus according to the first embodiment of the present invention.

FIG. 4 is a flow chart illustrating an image reconstructing operation for performing a back projection every projection data different in the number of views in the X-ray CT apparatus according to the first embodiment of the present invention.

FIG. 5 is a flow chart showing the details of a pre-process.

FIG. 6 is a flow chart illustrating the details of a three-dimensional image reconstructing process.

FIG. 7 is a diagram depicting a conventional X-ray data acquisition method.

FIG. 8 is a diagram illustrating resolutions on the circumferences of circles at respective radii.

FIG. 9 is a diagram showing a case in which the number of views is changed for every channel position.

FIG. 10 is a diagram illustrating re-sampling of projection data on view numbers different for every channel position.

FIG. 11 is a diagram showing image reconstruction from divided projection data.

FIG. 12 is a diagram depicting data acquisition of respective view numbers and data acquisition of X-ray dosage correction channels corresponding thereto.

FIG. 13 is a diagram showing an example illustrative of X-ray dosage correction channels for respective view numbers in the X-ray detector.

FIG. 14 is a diagram illustrating X-ray dosage correction data of view numbers V3, V2, V1 divided from X-ray dosage correction channel data of a view number V_{LCM}.

FIG. 15 is a diagram showing an example illustrative of an X-ray dosage correction channel in the X-ray detector.

FIG. 16 is a diagram illustrating a maximum imaging view field and a set imaging view field in the X-ray CT apparatus.

FIG. 17 is a diagram showing ranges of the X-ray detector, which are necessary for a maximum imaging view field area and a set imaging view field area in the X-ray CT apparatus.

FIG. 18 is a diagram showing a case in which no subject exists outside the set imaging view field.

FIG. 19 is a diagram showing a case in which the number of views is set in accordance with the set imaging view field area.

FIG. 20 is a diagram illustrating each imaging view field area set to a heart nearby area.

FIG. 21 is a block diagram showing an X-ray CT apparatus according to a sixth embodiment.

FIG. 22 is an explanatory diagram illustrating rotation of an X-ray generator (X-ray tube) and a multi-row X-ray detector employed in the sixth embodiment.

FIG. 23 is a diagram showing a case in which an imaging view field area varies depending on a z-direction position.

FIG. 24 is a diagram illustrating optimization of view numbers for respective channels at imaging data of respective rows in the multi-row X-ray detector.

FIG. 25 is a flow chart showing optimization of view numbers for respective channels at imaging data of respective rows in the multi-row X-ray detector and a flow for imaging thereof.

FIG. 26 is a diagram showing optimization of view numbers for respective channels at a conventional scan (axial scan) or a cine scan and a helical scan.

FIG. 27 is a diagram illustrating a case in which the helical scan is performed.

FIG. 28 is a diagram depicting data conversion for CT value conversion.

FIG. 29 is a diagram showing a subject existence area as viewed in a z direction.

### DETAILED DESCRIPTION OF THE INVENTION

The present invention will hereinafter be explained in further detail by embodiments illustrated in the figures. Incidentally, the present invention is not limited to or by the embodiments.

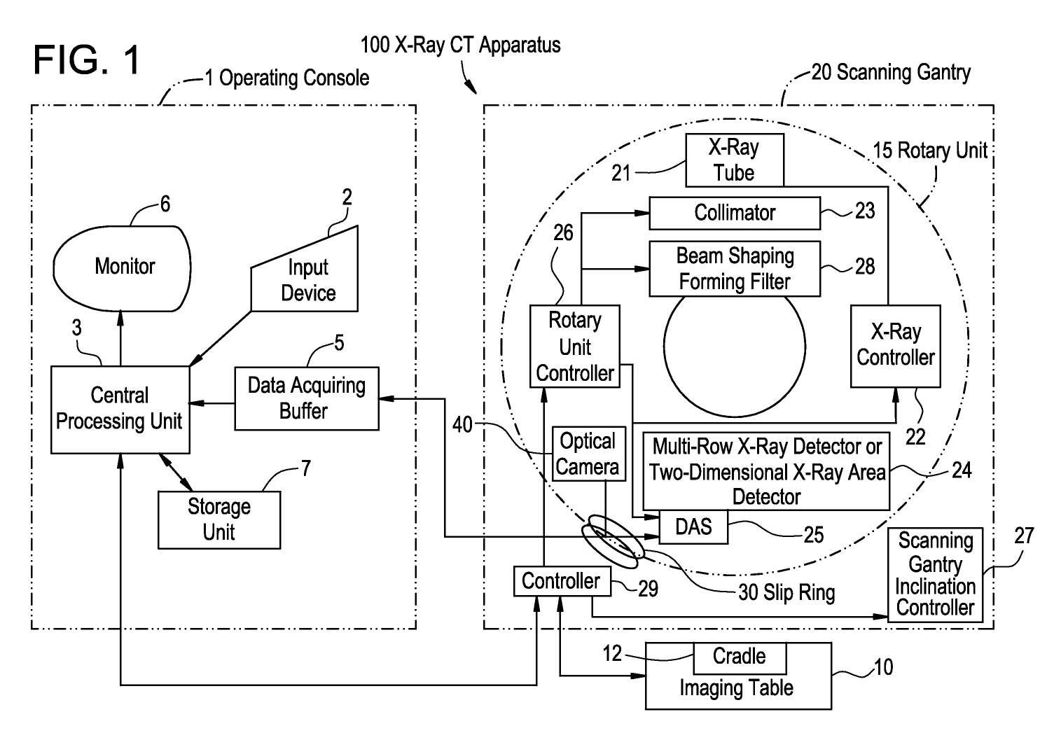

FIG. 1 is a configuration block diagram showing an X-ray CT apparatus according to a first embodiment of the present invention. The X-ray CT apparatus 100 is equipped with an operation console 1, an imaging or photographing table 10 and a scan gantry 20.

The operation console 1 includes an input device 2 which accepts an input from an operator, a central processing unit 3 which executes a pre-process, an image reconstructing process, a post-process, etc., a data acquisition buffer 5 which acquires or collects X-ray detector data acquired by the scan gantry 20, a monitor 6 which displays a tomographic image image-reconstructed from projection data obtained by pre-processing the X-ray detector data, and a storage device 7 which stores programs, X-ray detector data, projection data and X-ray tomographic images therein.

An input for imaging or photographing conditions is inputted from the input device 2 and stored in the storage device 7.

The photographing table 10 includes a cradle 12 which inserts and draws a subject into and from a bore or aperture of the scan gantry 20 with the subject placed thereon. The cradle 12 is elevated and moved linearly on the photographing table by a motor built in the photographing table 10.

The scan gantry 20 includes an X-ray tube 21, an X-ray controller 22, a collimator 23, an X-ray beam forming filter 28, a multi-row X-ray detector 24, a DAS (Data Acquisition System) 25, a rotating section controller 26 which controls the X-ray tube 21 or the like rotated about a body axis of the subject, and a control controller 29 which swaps control signals or the like with the operation console 1 and the photographing table 10. The X-ray beam forming filter 28 is an X-ray filter configured so as to be thinnest in thickness as viewed in the direction of X-rays directed to the center of rotation corresponding to the center of imaging, to increase in thickness toward its peripheral portion and to be able to further absorb the X rays. Therefore, the body surface of a subject whose sectional shape is nearly circular or elliptic can be less exposed to radiation. The scan gantry 20 can be tiled about ±30° or so forward and rearward as viewed in the z direction by a scan gantry tilt controller 27.

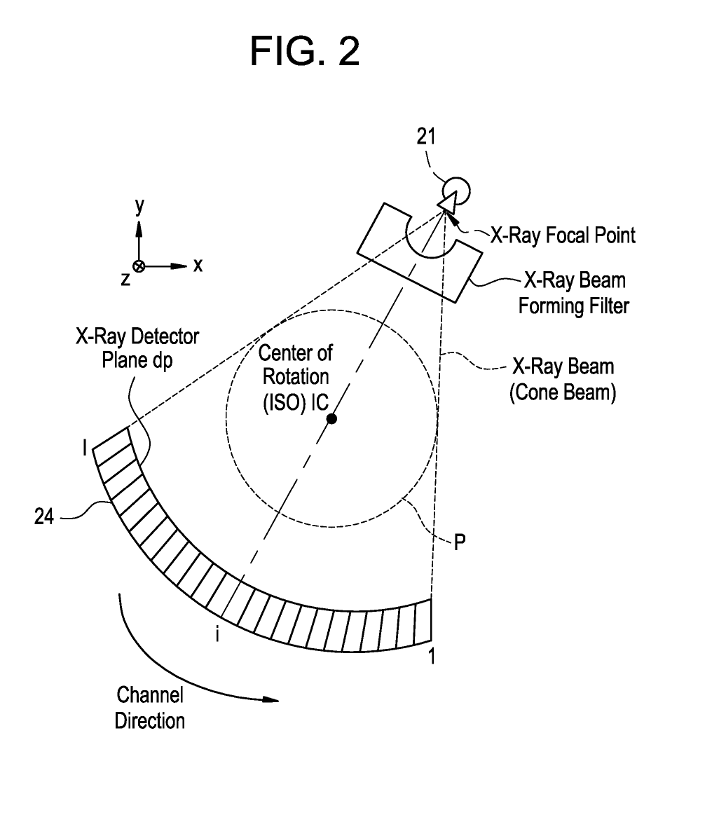

FIG. 2 is a diagram for describing a geometrical arrangement or layout of the X-ray tube 21 and the multi-row X-ray detector 24.

The X-ray tube 21 and the multi-row X-ray detector 24 are rotated about the center of rotation IC. Assuming that the vertical direction is a y direction, the horizontal direction is an x direction and the travel direction of the table orthogonal to these is a z direction, the plane at which the X-ray tube 21 and the multi-row X-ray detector 24 are rotated, is an xy plane. The direction, in which the cradle 12 is moved, corresponds to the z direction.

The X-ray tube 21 generates an X-ray beam called a cone beam CB. When the direction of a central axis of the cone beam CB is parallel to the y direction, this is defined as a view angle 0°.

The multi-row X-ray detector 24 has X-ray detector rows corresponding to 256 rows, for example. Each of the X-ray detector rows has X-ray detector channels corresponding to 1024 channels, for example.

X-rays are applied and acquired projection data are A/D converted by the DAS 25 from the multi-row X-ray detector 24, which in turn are inputted to the data acquisition buffer 5 via a slip ring 30. The data inputted to the data acquisition buffer 5 are processed by the central processing unit 3 in accordance with the corresponding program stored in the storage device 7, so that the data are image-reconstructed as a tomographic image, followed by being displayed on the monitor 6.

In the present invention, X-ray detector data or projection data corresponding to a plurality of types of view numbers different according to the channel positions are acquired and image-reconstructed as a tomographic image.

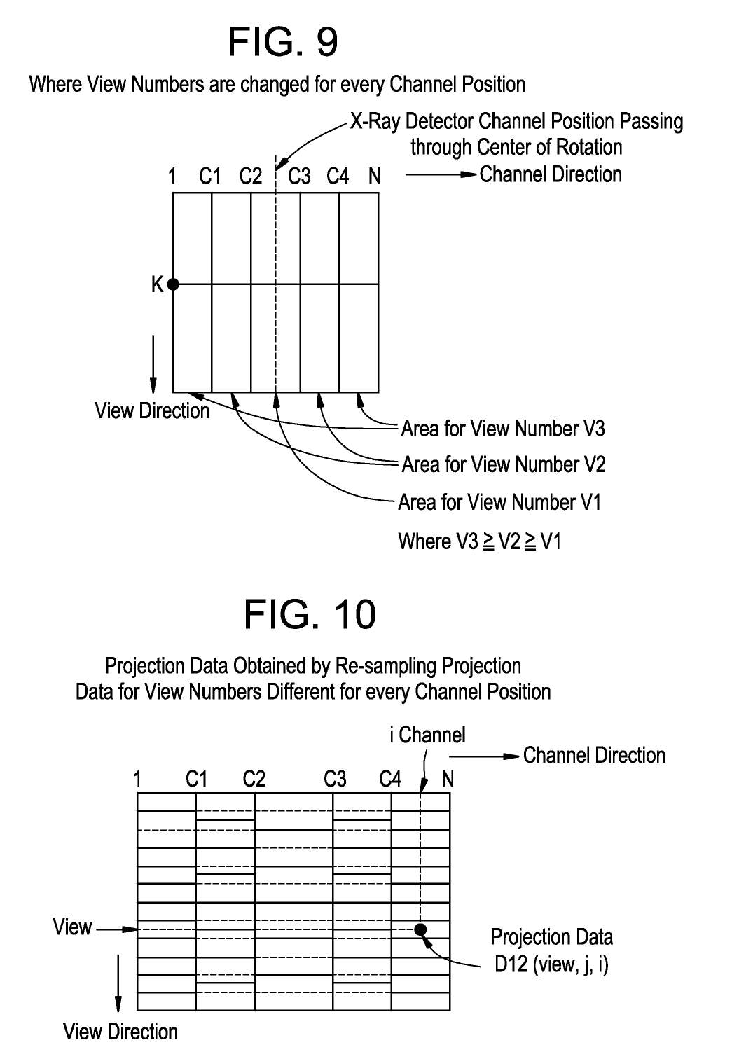

FIG. 9 shows X-ray detector data at the time that the number of views is changed for every channel position.

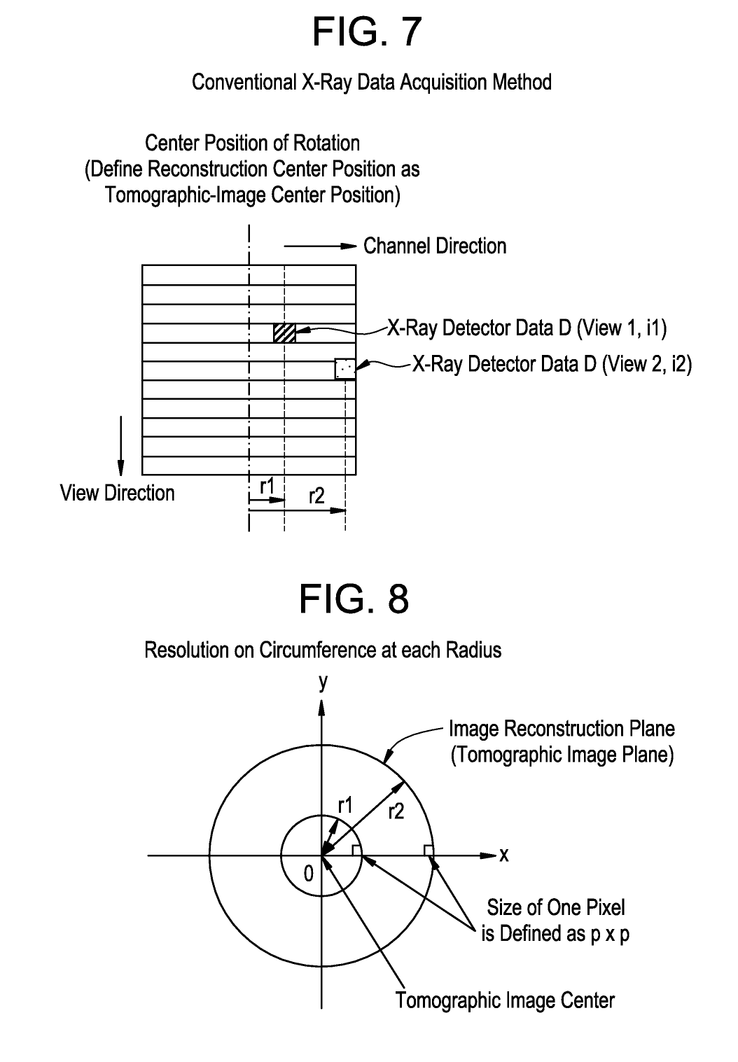

FIG. 9 shows X-ray detector data or projection data of X-ray detectors corresponding to one row in a manner similar to FIG. 7. The horizontal axis indicates a channel direction for X-ray detector data or projection data, and the vertical axis indicates a view direction for the X-ray detector data and the projection data.

X-ray detector data from a 1 channel to a C1-1 channel, X-ray detector data from a C1 channel to a C2-1 channel, X-ray detector data from a C2 channel to a C3-1 channel, X-ray detector data from a C3 channel to a C4-1 channel and X-ray detector data from a C4 channel to an N channel are respectively X-ray data-acquired at a view number V3, a view number V2, a view number V1, a view number V2 and a view number V3 over 360°. However, the relationship of magnitude between the view numbers is assumed to be V3≧V2≧V1.

When N=1000 (channels), for example, the following combinations are considered:

(1) C1=200, C2=400, C3=600, C4=800, V3=1500, V2=1000, V1=500

(2) C1=200, C2=450, C3=550, C4=800, V3=1500, V2=1000, V1=500

(3) C1=300, C2=450, C3=550, C4=700, V3=1500, V2=1000, V1=500

As a method of image-reconstructing the X-ray detector data, there are considered two image reconstructing methods shown below. Embodiments showing the following two cases will be explained below.

(1) A pre-process is executed while remaining at the view numbers different for every channel. Upon a reconstruction function convolution process and a backprojection process, the X-ray detector data at the view numbers V2 and V1 are re-sampled at the view number V3, and the X-ray detector data are subjected to the reconstruction function convolution process and the backprojection process after the view number is set to V3 with respect to all the channels.

(2) A pre-process is executed while remaining at view numbers different for every channel. Upon a reconstruction function convolution process and a backprojection process, X-ray detector data is separated into projection data different in view number in projection data space, which are separately subjected to the reconstruction function convolution process and the backprojection process respectively, thereby finally resulting in one tomographic image by a weighted addition process in image space.

### First Embodiment

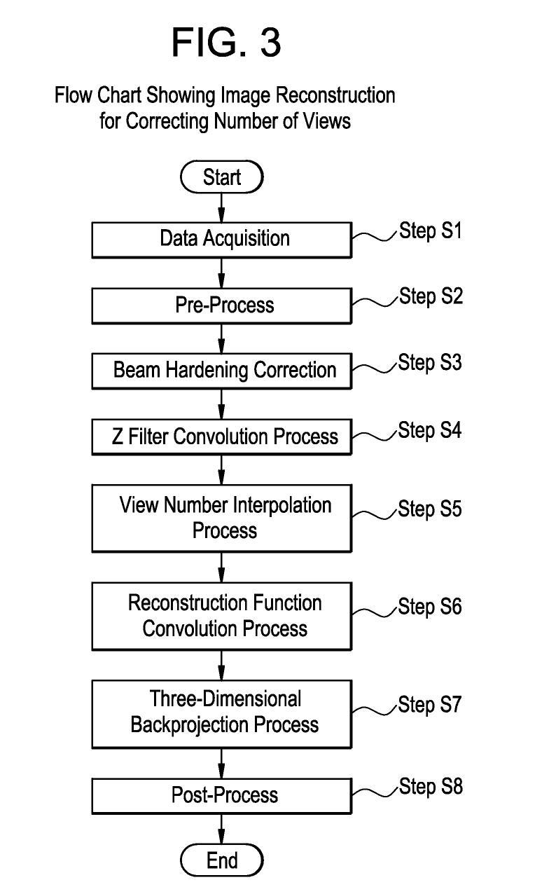

FIG. 3 is a flow chart showing the outline of the operation of the X-ray CT apparatus 100 according to the present invention.

At Step S1, the operation of rotating the X-ray tube 21 and the multi-row X-ray detector 24 about the subject and effecting data acquisition of X-ray detector data on the cradle 12 placed on the imaging or photographing table 10 while the table is being linearly moved, is performed upon a helical scan. Then a table linear movement z-direction position Ztable(view) is added to X-ray detector data D0(view, j, i) indicated by a view angle view, a detector row number j and a channel number i, thereby acquiring the X-ray detector data. Upon a conventional scan (axial scan) or a cine scan, the data acquisition system is rotated once or plural times while the cradle 12 placed on the photographing table 10 is being fixed to a given z-direction position, thereby to perform data acquisition of X-ray detector data. The cradle 12 is moved to the next z-direction position if necessary and thereafter the data acquisition system is rotated once or plural times again to perform data acquisition of X-ray detector data.

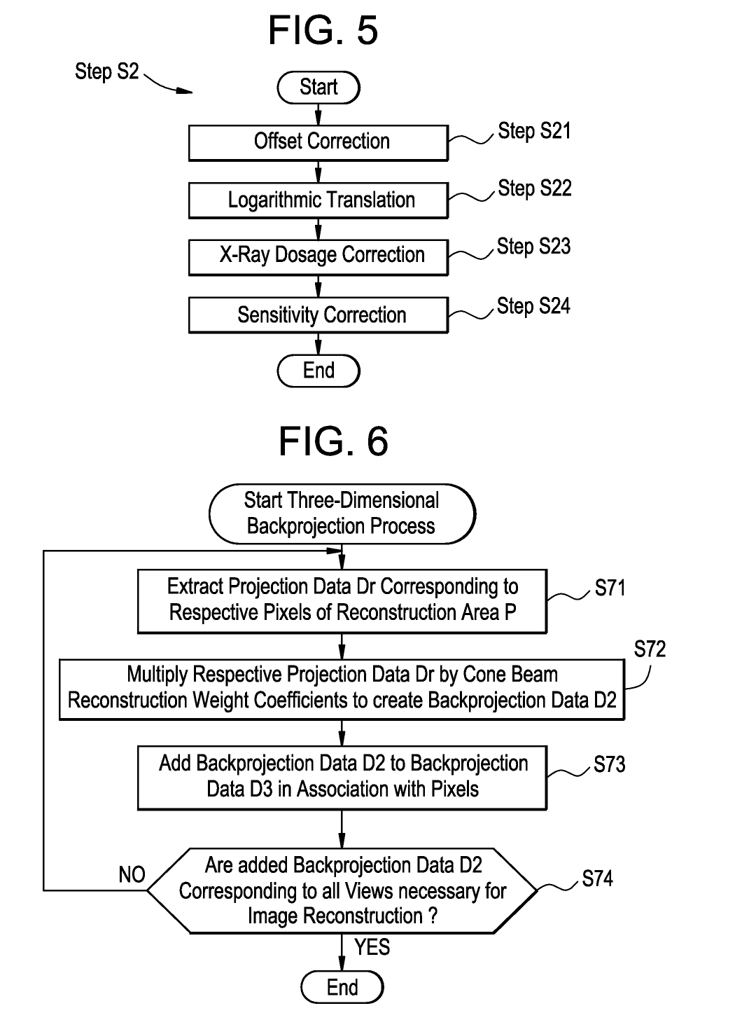

At Step S2, a pre-process is performed on the X-ray detector data D0(view, j, i) to convert it into projection data. As shown in FIG. 5, the pre-process comprises a Step S21 offset correction, Step S22 logarithmic translation, a Step S23 X-ray dosage correction and a Step S24 sensitivity correction.

Incidentally, there is a need to create X-ray dosage correction data for the view numbers V1, V2 and V3 in X-ray dosage correction channels for X-ray dosage correction. This will be explained later.

At Step S3, a beam hardening correction is effected on the pre-processed projection data D1(view, j, i). Assuming that upon the beam hardening correction S3, projection data subjected to the sensitivity correction S24 at the pre-process S2 is defined as D1(view, j, i) and data subsequent to the beam hardening correction S3 is defined as D11(view, j, i), the beam hardening correction S3 is expressed in the form of, for example, a polynomial as shown below.
D11(view,j,i)=D1(view,j,i)·(Bo(j,i)+B₁(j,i)·D1(view,j,i)+B₂(j,i)·D1(view,j,i)²) [Equation 1]

At Step S4, a z-filter convolution process for applying filters in the z direction (row direction) is effected on the projection data D11(view, j, i) subjected to the beam hardening correction.

At Step S4, after the pre-process at each view angle and each data acquisition system, projection data of the multi-row X-ray detector D11(view, j, i) (where i=1 to CH and j=1 to ROW) subjected to the beam hardening correction is multiplied by filters in which the following row-direction filter sizes are five rows, for example, in the row direction.

$$( {w}_{1} ( j ) , {w}_{2} ( j ) , {w}_{3} ( j ) , {w}_{4} ( j ) , {w}_{5} ( j ) ) \\ where {\sum }_{k = 1}^{5} {w}_{k} ( j ) = 1 \qquad [ Equation 2 ]$$

The corrected detector data D12(view, j, i) is expressed as follows:

$$D 12 ( view , j , i ) = {\sum }_{k = 1}^{5} ( D 11 ( view , j - k - 3 , i ) \cdot {w}_{k} ( j ) ) \qquad [ Equation 3 ]$$

Incidentally, assuming that the maximum value of the channel is CH and the maximum value of the row is ROW, the following equations are established.
D11(view,−1,i)=D11(view,O,i)=D11(view,1,i)
D11(viw,ROW,i)=D11(view,ROW+1,i)=D11(view,ROW+2,i) [Equation 4]

When row-direction filter coefficients are changed for every channel, slice thicknesses can be controlled depending upon the distance from an image reconstruction center. In a tomographic image, its peripheral portion generally becomes thick in slice thickness than the reconstruction center thereof. Therefore, the row-direction filter coefficients are optimally changed at the central and peripheral portions so that the slice thicknesses can also be made close to each other uniformly even at the peripheral portion and the image reconstruction center.

In the view number interpolation process of Step S5, interpolation is done on projection data space at parts for the view numbers V2 and V1 in order to re-sample projection data in match with V3 most large in view number, of the view numbers V3, V2 and V1 corresponding to the respective channel positions of the projection data shown in FIG. 9.

That is, the parts for the view number V3 are defined as projection data set every 360/V3°. On the other hand, the parts for the view numbers V2 and V1 are defined as projection data set every 360/V2° and 360/V1°.

As shown in FIG. 10, projection data set every fine 360/V3° are provided at the outer channel ranges [1, C1-1] and [C4, N].

On the other hand, projection data set every 360/N2° are provided at the inner channel ranges [C1, C2-1] and [C3, C4-1]. Further, projection data set every 360/N1° is provided at the inner channel range [C2, C3-1].

The range for [C1, C4-1] is interpolated into data set every 360/N3° as seen in the view direction to re-sample data. Determining data corresponding to a kth view at [1, C1-1] and [C4, N] from the projection data of [C1, C2-1], [C3, C4-1] or [C2, C3-1], for example, by linear interpolation yields the following. However, the projection data obtained by correction is assumed to be D12(view, j, i), and view, j and i are respectively assumed to be a view number, a row number and a channel number.

Assuming that the projection data at the channel range of [C1, C2-1] or [C3, C4-1] is defined as B(view, j, i) and the projection data at the channel range of [C2, C3-1] is defined as C(view, j, i), the projection data D12(k, j, i) at the kth view is given as shown below in the channel range of [C1, C2-1] or [C3, C4-1]

$$D 12 ( k , j , i ) = ( int ( k \cdot \frac{V 2}{V 3} ) + 1 - k \cdot \frac{V 2}{V 3} ) \cdot B ( int ( k \cdot \frac{V 2}{V 3} , j , i ) + ( k \cdot \frac{V 2}{V 3} - int ( k \cdot \frac{V 2}{V 3} ) ) \cdot B ( int ( k \cdot \frac{V 2}{V 3} ) + 1 , j , i ) \qquad [ Equation 5 ]$$

Also the projection data is given as follows in the channel range of [C2, C3-1].

$$D 12 ( k , j , i ) = ( int ( k \cdot \frac{V 1}{V 3} ) + 1 - k \cdot \frac{V 1}{V 3} ) \cdot C ( int ( k \cdot \frac{V 1}{V 3} , j , i ) + ( k \cdot \frac{V 1}{V 3} - int ( k \cdot \frac{V 1}{V 3} ) ) \cdot C ( int ( k \cdot \frac{V 1}{V 3} ) + 1 , j , i ) \qquad [ Equation 6 ]$$

Thus, the projection data B(view, j, i) and C(view, j, i) are interpolated to create projection data D12(view, j, i) equivalent to the V3 view corresponding to one rotation in a range corresponding to all channel ranges [1, N]. The subsequent reconstruction function convolution process and three-dimensional backprojection process are advanced as usual with all the channels as the projection data for the V3 view.

At Step S6, the reconstruction function convolution process is performed. That is, projection data is subjected to Fourier transformation and multiplied by a reconstruction function, followed by being subjected to inverse Fourier transformation. Assuming that upon the reconstruction function convolution process S5, data subsequent to the z filter convolution process is defined as D12, data subsequent to the reconstruction function convolution process is defined as D13, and the convoluting reconstruction function is defined as Kernel(j), the reconstruction function convolution process is expressed as follows:
D13(view,j,i)=D12(view,j,i)*Kernel(j) [Equation 7]

At Step S7, a three-dimensional backprojection process is effected on the projection data D13(view, j, i) subjected to the reconstruction function convolution process to determine backprojection data D3(x, y). An image to be image-reconstructed is three-dimensionally image-reconstructed on a plane, i.e., an xy plane orthogonal to the z axis. A reconstruction area or plane P to be shown below is assumed to be parallel to the xy plane. The three-dimensional backprojection process will be explained later referring to FIG. 6.

At Step S8, a post-process including image filter convolution, CT value conversion and the like is effected on backprojection data D3(x, y, z) to obtain a CT or tomographic image D31(x, y).

While the process for the CT value conversion is included in the post-process at Step S8, a backprojected image D3(x, y) is data-converted into CT values of air-1000(HU) and water 0(HU) upon the CT value conversion.

Assuming that a backprojected value is defined as P=D3(x, y) and image data subsequent to the CT value conversion is defined as Q=D31(x, y), data conversion for the CT value conversion is expressed as given below and varies depending upon backprojected view numbers.

CT value data conversion function for view number Vₐ fₐ: Q=fₐ(P)

CT value data conversion function for view number V_{b} f_{b}: Q=f_{b}(P)

CT value data conversion function for view number V_{c} f_{c}: Q=f_{c}(P)

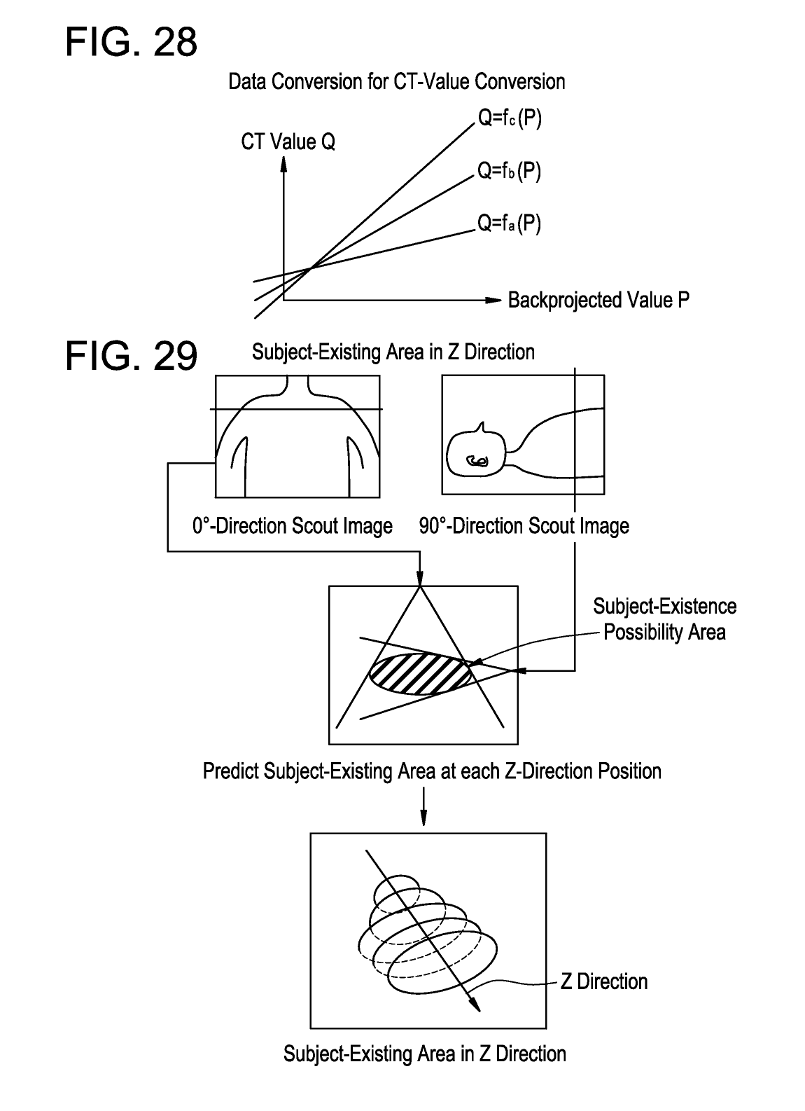

As shown in FIG. 28, fₐ, f_{b} and f_{c} are expressed in linear function form as follows:

CT value data conversion function for view number Vₐ Q=Kₐ·P+Cₐ

CT value data conversion function for view number V_{b} Q=K_{b}·P+C_{b}

CT value data conversion function for view number V_{c} Q=K_{c}·P+C_{c}

Assuming that upon the image filter convolution process in the post-process, a tomographic image subsequent to the three-dimensional backprojection is defined as D31(x, y, z), data subsequent to the image filter convolution is defined as D32(x, y, z), and an image filter is defined as Filter(z), the following equation is established.
D32(x,y,z)=D31(x,y,z)*Filter(z) [Equation 8]

That is, since the independent image filter convolution processes can be carried out every j row of detector, the difference between noise characteristics set every row and the difference between resolution characteristics set every row can be corrected. The resultant tomographic image is displayed on the monitor 6.

FIG. 6 is a flow chart showing the three-dimensional backprojection process (Step S7 in FIG. 5). In the present embodiment, an image to be image-reconstructed is three-dimensionally image-reconstructed on a plane, i.e., an xy plane orthogonal to the z axis. The following reconstruction area P is assumed to be parallel to the xy plane.

At Step S71, attention is given to one of all views (i.e., views corresponding to 360° or views corresponding to “180°+fan angles”) necessary for image reconstruction of a tomographic image. Projection data Dr corresponding to respective pixels in a reconstruction area P are extracted.

A square area of 512×512 pixels, which is parallel to the xy plane, is assumed to be a reconstruction area P. If projection data on lines T0 through T511 obtained by projecting a pixel row L0 parallel to an x axis of y=0 to a pixel row L511 of y=511 on the plane of the multi-row X-ray detector 24 in an X-ray penetration direction are extracted from the pixel row L0 to the pixel row L511, then they result in projection data Dr(view, x, y) backprojected on the respective pixels on the tomographic image. However, x and y correspond to the respective pixels (x, y) of the tomographic image.

The X-ray penetration direction is determined depending on geometrical positions of the X-ray focal point of the X-ray tube 21, the respective pixels and the multi-row X-ray detector 24. Since, however, the z coordinates z(view) of X-ray detector data D0(view, j, i) are known with being added to X-ray detector data as a table linear movement z-direction position Ztable(view), the X-ray penetration direction can be accurately determined within the X-ray focal point and the data acquisition geometrical system of the multi-row X-ray detector even in the case of the X-ray detector data D0(view, j, i) placed under acceleration and deceleration.

Incidentally, when some of lines are placed out of the multi-row X-ray detector 24 as viewed in the channel direction as in the case of, for example, the line T0 obtained by projecting the pixel row L0 on the plane of the multi-row X-ray detector 24 in the X-ray penetration direction, the corresponding projection data Dr(view, x, y) is set to “0”. When it is placed outside the multi-row X-ray detector 24 as viewed in the z direction, the corresponding projection data Dr(view, x, y) is determined as extrapolation.

Thus, the projection data Dr (view, x, y) corresponding to the respective pixels of the reconstruction area P can be extracted.

Referring back to FIG. 6, at Step S72, the projection data Dr(view, x, y) are multiplied by a cone beam reconstruction weight coefficient to create projection data D2(view, x, y).

Now, the cone beam reconstruction weight function w(i, j) is as follows. Generally, when the angle which a linear line connecting the focal point of the X-ray tube 21 and a pixel g(x, y) on the reconstruction area P (xy plane) at view=βa forms with a center axis Bc of an X-ray beam is assumed to be γ and its opposite view is assumed to be view=βb in the case of fan beam image reconstruction, the following equation is established.
βb=βa+180°−2γ [Equation 9]

When the angles which the X-ray beam passing through the pixel g(x, y) on the reconstruction area P and its opposite X-ray beam form with the reconstruction plane P, are assumed to be αa and αb, they are multiplied by con beam reconstruction weight coefficients ωa and ωb dependant on these and added together to determine backprojection pixel data D2(0, x, y) in the following manner.
D2(0,x,y)=ωa·D2(0,x,y)_{—}a+ωb·D2(0,x,y)_{—}b [Equation 10]

where D2(0,x,y)_a indicates projection data for the view βa, and D2(0,x,y)_b indicates projection data for the view βb.

Incidentally, the sum of the con beam reconstruction weight coefficients corresponding to the beams opposite to each other is as follows:
ωa+ωb=1 [Equation 11]

The above addition with multiplication of the cone beam reconstruction weight coefficients ωa and ωb enables a reduction in cone angle archfact.

In the case of the fan beam image reconstruction, each pixel on the reconstruction area P is multiplied by a distance coefficient. Assuming that the distance from the focal point of the X-ray tube 21 to each of the detector row j and channel i of the multi-row X-ray detector 24 corresponding to the projection data Dr is r0, and the distance from the focal point of the X-ray tube 21 to each pixel on the reconstruction area P corresponding to the projection data Dr is r1, the distance coefficient is given as (r1/r2)².

In the case of parallel beam image reconstruction, each pixel on the reconstruction area P may be multiplied by the cone beam reconstruction weight coefficient w(i, j) alone.

At Step S73, the projection data D2(view, x, y) is added to its corresponding backprojection data D3(x, y) cleared in advance in association with each pixel.

At Step S74, Steps S61 through S63 are repeated with respect to all the views (i.e., views corresponding to 360° or views corresponding to “180°+fan angles”) necessary for image reconstruction of the tomographic image to obtain backprojection data D3(x, y).

Incidentally, the reconstruction area P may be set as a circular area whose diameter is 512 pixels, without setting it as the square area of 512×512 pixels.

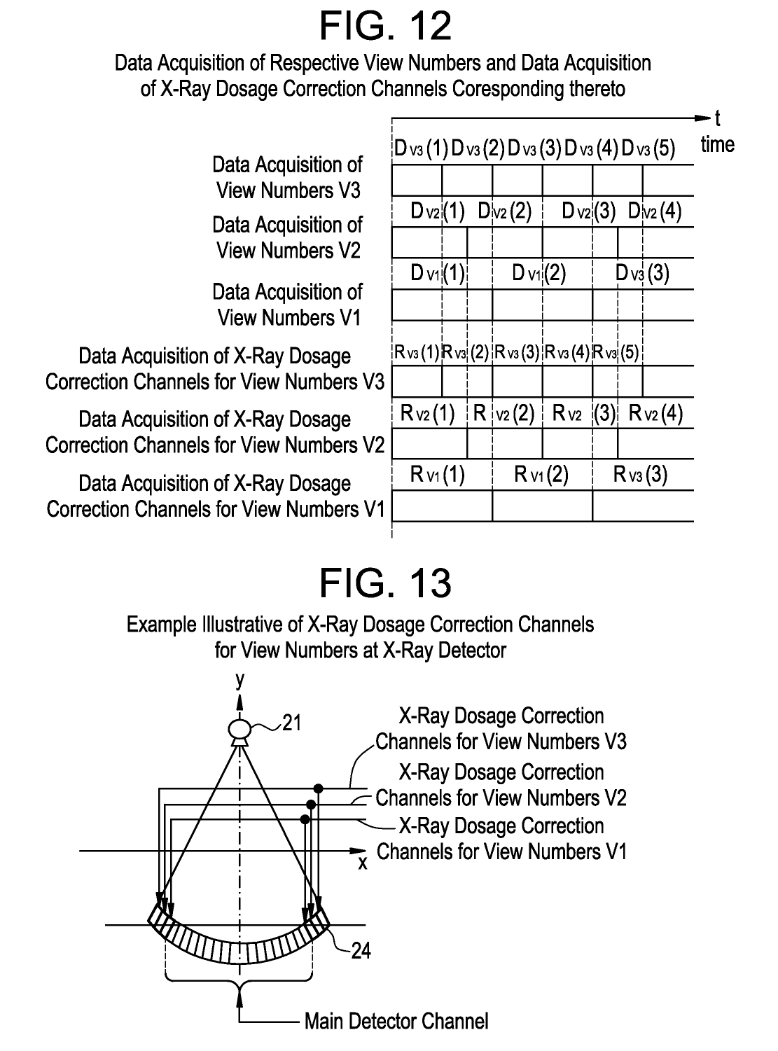

When the X-ray dosage correction is effected on X-ray detector data for view numbers different from V1, V2 and V3 or projection data for every channel position as shown in FIG. 9 upon the X-ray dosage correction of Step S23 placed prior to Step S2, X-ray dosage correction channels synchronized with the respective view numbers of V1, V2 and V3 are required. In this case, X-ray dosage correction channels for the view numbers V3, V2 and V1 identical in data acquisition timing are required in association with data acquisition for the view number V3, data acquisition for the view number V2 and data acquisition for the view number V1 as shown in FIG. 12. In this case, two methods are considered.

(1) Three types of X-ray dosage correction channels for V3, V2 and V1 are respectively prepared.

(2) One type of X-ray dosage correction channel for the view number of the least common multiple V_{LCM} of V3, V2 and V1 is prepared and allotted to the view numbers V3, V2 and V1.

In the case of (1), as shown in FIG. 13, the X-ray dosage correction channels for the respective view numbers are prepared one by one or plural by plural at both ends or one side of the multi-row X-ray detector 24. The following X-ray dosage correction channel data are acquired or collected from these channels.

X-ray dosage correction channel data for view number V3: R_{V3}(view)

X-ray dosage correction channel data for view number V2: R_{V2}(view)

X-ray dosage correction channel data for view number V1: R_{V1}(view)

Upon the X-ray dosage correction, the following data are corrected based on the above X-ray dosage correction channel data R_{V3}(view), R_{V2}(view) and R_{V1}(view).

X-ray detector data for view number V3: D_{V3}(view)

X-ray detector data for view number V2: D_{V2}(view)

X-ray detector data for view number V1: D_{V1}(view)

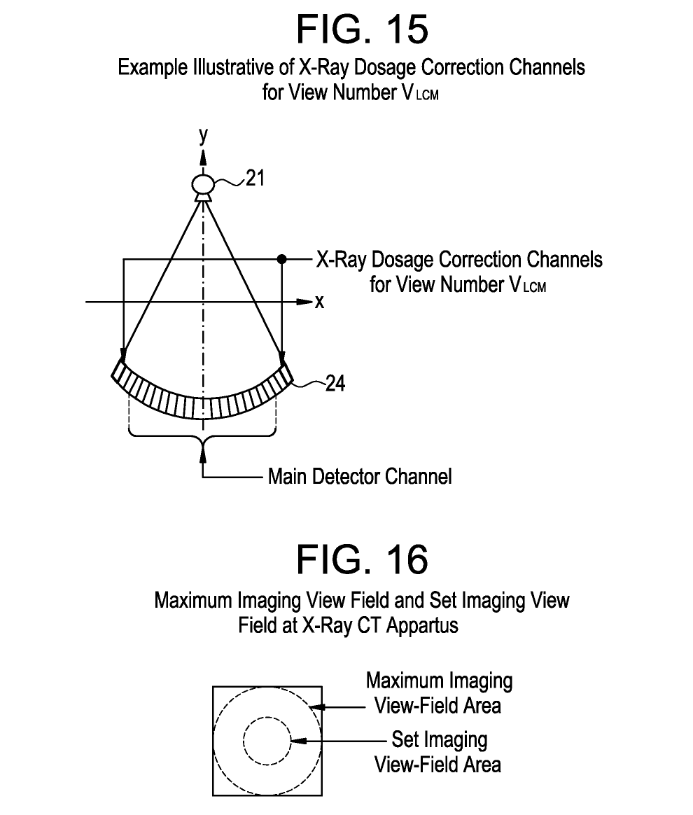

In the case of (2), as shown in FIG. 15, an X-ray dosage correction channel for a view number V_{LCM} is prepared at least one by one at both ends of the multi-row X-ray detector 24 or at least one on one side thereof. The following X-ray dosage correction channel data are determined by division from the X-ray dosage correction channel data. They are as follows:

X-ray dosage correction channel data for view number V3: R_{V3}(view)

X-ray dosage correction channel data for view number V2: R_{V2}(view)

X-ray dosage correction channel data for view number V1: R_{V1}(view)

X-ray dosage correction channel data for view number V_{LCM}: R_{VLCM}(view).

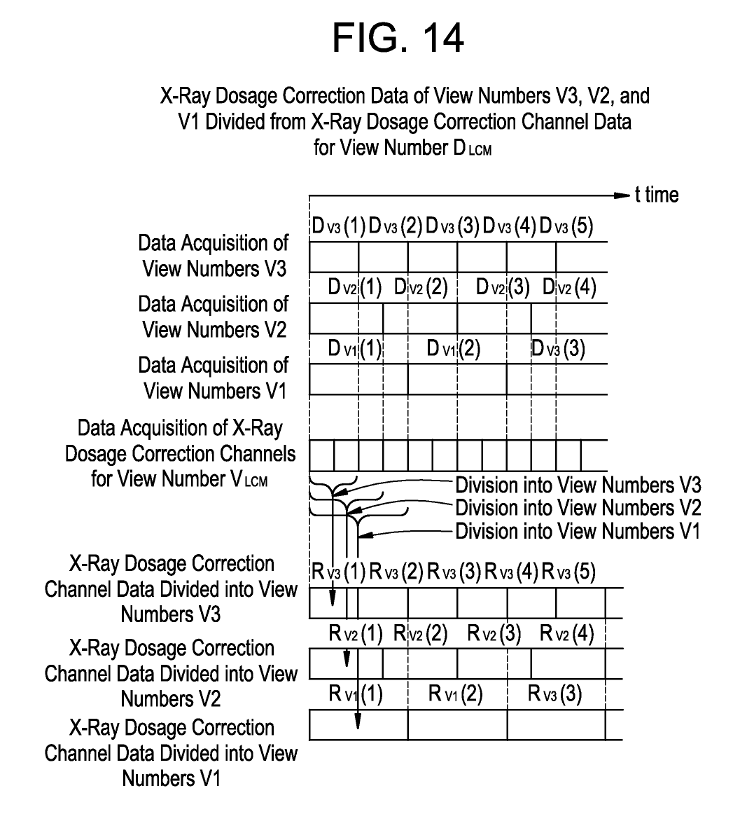

When the two division of the view number V_{LCM} is a view V3, the three division of the view number V_{LCM} is a view V2 and the four division of the view number V_{LCM} is a view V1 as shown in FIG. 14, following equations are obtained.
R_{V3}(view)=R_{VLCM}(2·view)+R_{VLCM}(2·view+1)
R_{V2}(view)=R_{VLCM}(3·view)+R_{VLCM}(3·view+2)+R_{VLCM}(3·view+3)
R_{V1}(view)=R_{VLCM}(4·view)+R_{VLCM}(4·view+1)+R_{VLCM}(4·view+2)+R_{VLCM}(4·view+3) [Equation 12]

R_{V3}(view), R_{V2}(view) and R_{V1}(view) may be determined by division in the above-described manner.

Upon the X-ray dosage correction, the following data are corrected based on the above X-ray dosage correction channel data R_{V3}(view), R_{V2}(view) and R_{V1}(view).

X-ray detector data D_{V3}(view) for view number V3

X-ray detector data D_{V2}(view) for view number V2

X-ray detector data D_{V1}(view) for view number V1

### Second Embodiment

In the above first embodiment, the X-ray detector data or projection data for the view numbers V2 and V1 are interpolated in the view direction to re-sample the X-ray detector data or projection data for the view numbers V2 and V1 at the view number V3 and converted to the X-ray detector data or projection data for the view number V3, whereby the image reconstruction is carried out.

However, a second embodiment to be described below is a method for image-reconstructing X-ray detector data or projection data for view numbers V3, V2 and V1 without the fear of degradation in resolution of data in a view direction due to view-direction interpolation and degradation in resolution in an xy plane on a tomographic image and without performing the interpolation in the view direction.

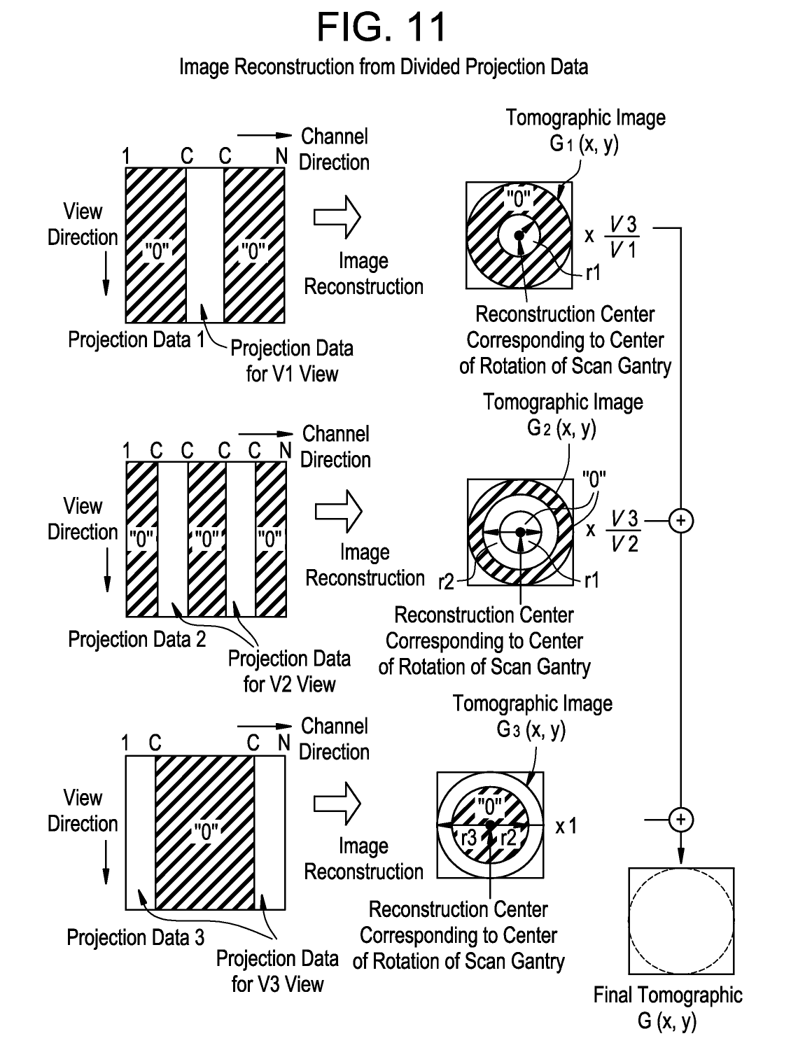

Conceptually, the X-ray detector data or projection data different in view number depending on channel ranges, i.e., the projection data of FIG. 9 subsequent to the pre-process is divided into three projection data 1, 2 and 3 as shown in FIG. 11 as in the case in which as shown in FIG. 9, the channel ranges [1, C1-1] and [C4, N] are defined as the V3 view, the channel ranges [C1, C2-1] and [C3, C4-1] are defined as the V2 view and the channel range [C2, C3-1] is defined as the V1 view. A reconstruction function convolution process and a three-dimensional backprojection process are effected on the respective projection data to perform image reconstruction thereof. The image-reconstructed tomographic images are multiplied by weight coefficients of “V3/V1”, “V3/V2” and “1” to perform a weighted addition process, followed by being formed as a final tomographic image.

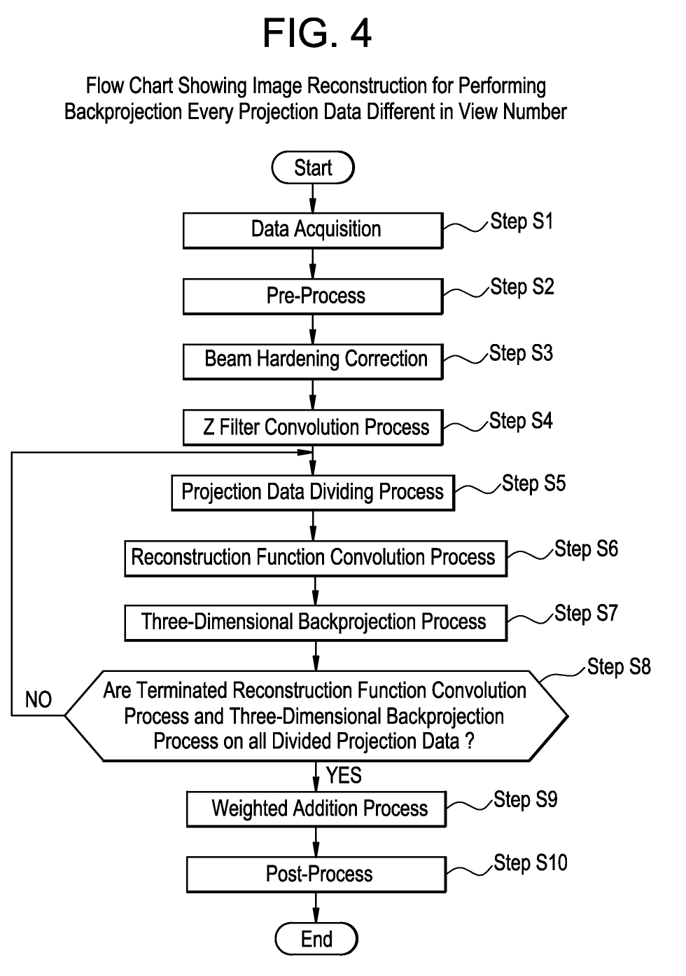

A flow for processing will be explained below in accordance with a flow chart shown in FIG. 4.

At Step S1, data acquisition is performed.

At Step S2, a pre-process is carried out.

At Step S3, a beam hardening correction is performed.

At Step S4, a z filter convolution process is carried out.

Steps S1 to S4 may be similar to the process of the first embodiment shown in FIG. 3.

At Step S5, a projection data dividing process is performed.

At Step S5, as shown in FIG. 11, the projection data is divided and extracted for every channel range different in view number for the projection data. Thereafter, projection data values “0” are embedded into the channel ranges free of the projection data as shown in FIG. 11, and the projection data is separated into projection data corresponding to types of different view numbers. Since there are shown three types of view numbers in FIG. 11, the projection data is separated into three types of projection data.

At Step S6, a reconstruction function convolution process is performed.

At Step S7, a three-dimensional backprojection process is carried out.

Steps S6 and S7 may be similar to the process of the first embodiment shown in FIG. 3.

At Step S8, it is determined whether the reconstruction function convolution process and the three-dimensional backprojection process on all the divided projection data have been finished. If the answer is found to be YES, then the process flow proceeds to Step S9. If the answer is found to be NO, then the process flow is returned to Step S6.

At Steps S6 and S7, the reconstruction function convolution process and the three-dimensional backprojection process are repeated by the number of the projection data divided at Step S5, i.e., the types of view numbers different from one another. Since the three types of projection data are processed in FIG. 11, Steps S6 and S7 are repeated three times.

At Step S9, a weighted addition process is performed.

At Step S9, as shown in FIG. 11, the reconstruction function convolution process and the three-dimensional backprojection process are performed and the image-reconstructed individual tomographic images are multiplied by weight coefficients, whereby the weighted addition process is performed.

Assuming that the tomographic image image-reconstructed from the channel range [C2, C3-1] is given as G₁(x, y), the tomographic image image-reconstructed from the channel ranges [C1, C2-1] and [C3 C4-1], is given as G₂(x, y), the tomographic image image-reconstructed from the channel ranges [1, C1-1] and [C4, N] is given as G₃(x, y), and the final tomographic image is given as G(x, y), G(x, y) is expressed by the following equation:

$$G ( x , y ) = \frac{V 3}{V 1} \cdot {G}_{1} ( x , y ) + \frac{V 3}{V 2} \cdot {G}_{2} ( x , y ) + 1 \cdot {G}_{3} ( x , y ) \qquad [ Equation 13 ]$$

These weight coefficients “V3/V1”, “V3/V2” and “1” result from the difference between the view numbers at the time the three-dimensional backprojection is done.

A post-process is carried out at Step S10.

Step S10 may be similar to the process of the first embodiment shown in FIG. 3.

Thus, in the second embodiment, the interpolation is done on the projection data space in the view direction using the X-ray detector data or projection data different for every channel range. The reconstruction function convolution process is directly performed on the X-ray detector data or projection data different for every channel range without reducing the resolution of the projection data as seen in the view direction. Thereafter, the three-dimensional backprojection process is done, whereby the tomographic image free of degradation in the resolution in the view direction is obtained by the image reconstruction.

According to the X-ray CT apparatus or the X-ray CT image reconstructing method, as the effects of the present invention obtained in the above X-ray CT apparatus, there can be provided an X-ray CT apparatus which reduces the number of X-ray data acquisition views in a data acquisition system (DAS) 25 of an X-ray CT apparatus having a one-row type X-ray detector or an X-ray CT apparatus having a two-dimensional area X-ray detector of a matrix structure, which is typified by a multi-row X-ray detector or a flat panel X-ray detector, and which implements optimization of required performance and throughput capacity of the data acquisition system (DAS) 25.

### Third Embodiment

An X-ray CT apparatus makes an attempt to change a reconstruction function for every region of a subject. In this case, the reconstruction function ranges from a high-resolution reconstruction function to a relatively low-resolution reconstruction function. The reconstruction function is used for convolution in a channel direction of an X-ray detector. Since projection data corresponding to each pixel of a tomographic image, subjected to a reconstruction function convolution process in the channel direction of the X-ray detector is backprojected in the direction of 360°, spatial resolution on an xy plane in the tomographic image depends upon the reconstruction function. In this case, the optimum number of views is necessary for every channel position even for the purpose of avoiding degradation in the resolution in the circumferential direction such as shown in FIG. 8 at the peripheral portion of the tomographic image in particular.

That is, the high-resolution reconstruction function more needs the number of views. The relatively low-resolution reconstruction function needs not to increase the number of views so much. In consideration of such points, the view number V3, view number V2 and view number V1 and the switching channel positions C1, C2, C3 and C4 for the view numbers, which are shown in FIG. 9, can be optimized depending upon the reconstruction functions.

### Fourth Embodiment

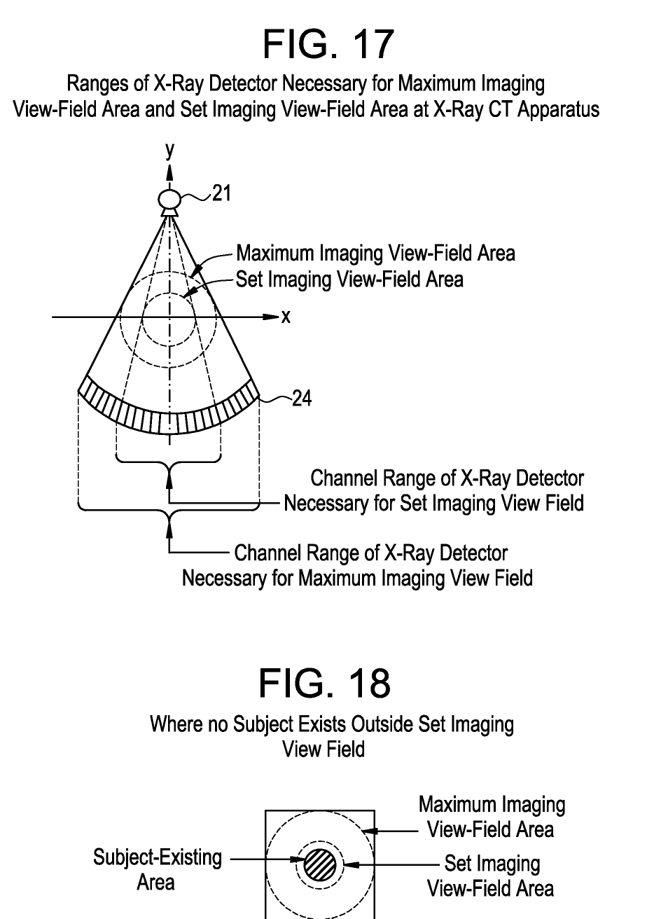

In an X-ray CT apparatus, an imaging view field is set for every region of a subject as shown in FIG. 16. X-ray detector channel ranges necessary for the set imaging view field are given as shown in FIG. 17. Data corresponding to sufficiently required view numbers may be acquired through some X-ray detector channels of X-ray detector channels necessary for the maximum imaging view field.

Particularly when a subject is sufficiently within the set imaging view field as shown in FIG. 18 and only air exists outside the set imaging view field, X-ray data may not be acquired in areas placed there outside or the number of views may be reduced. As to X-ray detector data or projection data in this case, a view number V1 enough to avoid degradation in spatial resolution is set in a channel range of [C1, C2-1] which covers the set imaging view field, and view numbers V3 may be extremely reduced in channel ranges of [1, C1-1] and [C2, N] corresponding to the areas placed outside the set imaging view field, or the view number may be set to V3=0.

Image reconstruction in this case may use the image reconstructing method according to the first embodiment or the image reconstructing method according to the second embodiment.

Thus, even when the subject-existing area is limited and only the neighborhood of the subject is set as the imaging view field, channel ranges A/D converted and processed by the corresponding data acquisition system (DAS) 25 can be set with efficiency.

### Fifth Embodiment

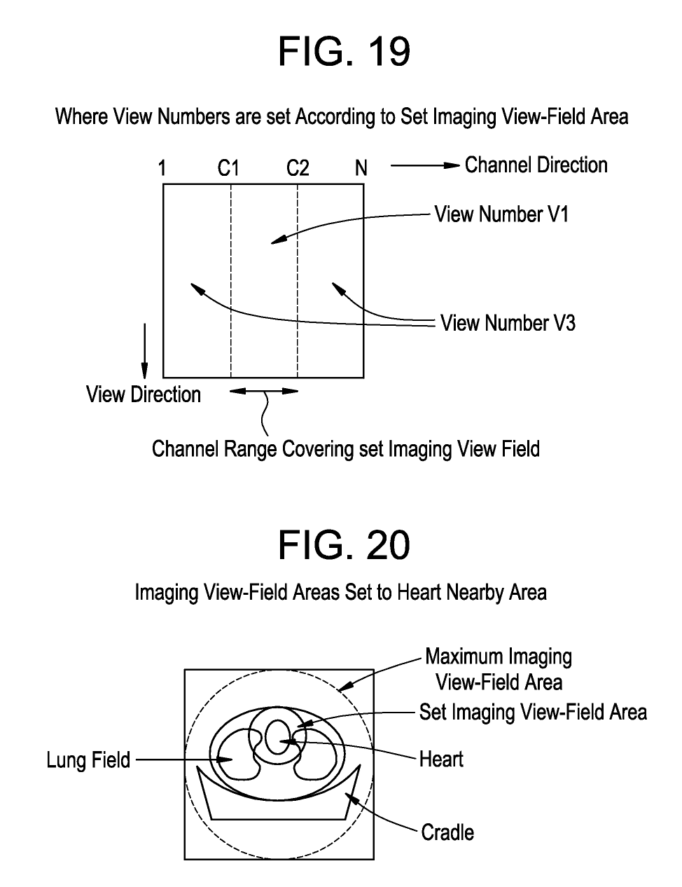

As in the case where the heart in each lug field is imaged or photographed as shown in FIG. 20, for example, an imaging view field is set to the neighborhood of the heart, and a view number V1 commensurate with pixel resolution of an area for the heart is set. In an area which includes a lug field or the like other than its heart area, X-ray data acquisition is performed at a view number V3 of such an extent that a pixel value (CT value) at an area in the vicinity of the boundary between the set imaging view field and an area placed there outside is not raised abnormally. As to X-ray detector data or projection data in this case, a channel range [C1, C2-1] which covers an imaging view field set to a heart nearby area, may be set, its view number may be defined as a V1 view and its outer view number may be defined as a V3 view in FIG. 19. In this case, V1≧V3 is established. Thus, the pixel value (CT value) at the boundary outside the set imaging view field is not increased either and the heart nearby area in the imaging view field set with sufficient spatial resolution can be imaged or photographed.

Thus, even when the subject exists outside the set imaging view field, the view numbers for the channel ranges placed outside the imaging view-field area set to such an extent as not to influence image quality in the set imaging view-field area may be defined.

Thus, the channel ranges of a data acquisition system (DAS) 25 and the view numbers for X-ray data acquisition can also be optimized in such a manner that no problem occurs in the image quality in the set imaging view-field area.

### Sixth Embodiment

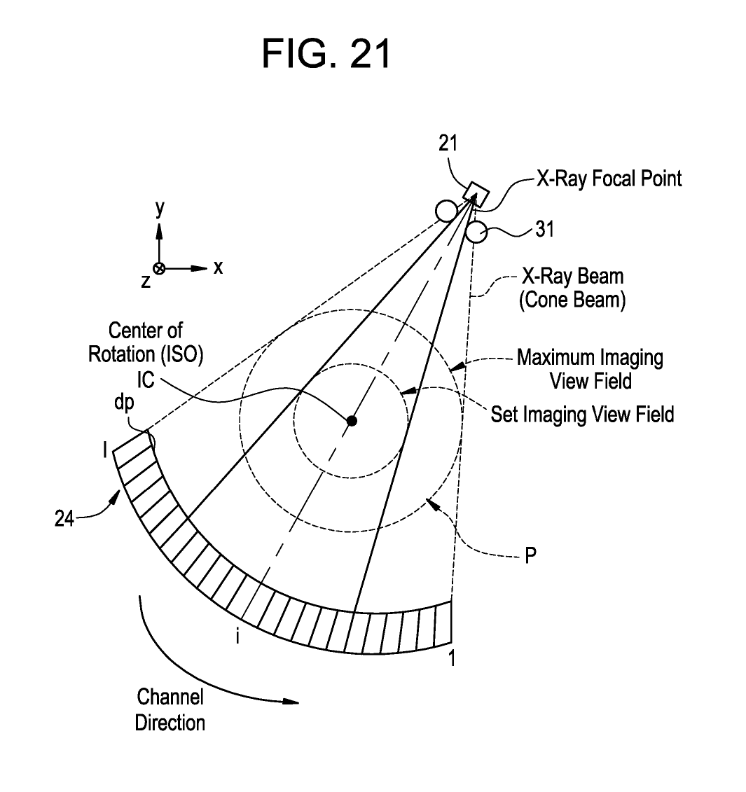

While X rays are applied to the full imaging view field as an X-ray exposure or irradiation area upon photography or imaging of the heart nearby area in the fifth embodiment, the X-ray irradiation area may also be limited only to an imaging view-field area to which X-ray irradiation is set by provision of a channel-direction collimator 31 as shown in FIG. 21 from the viewpoint of a reduction in X-ray exposure.

As to X-ray detector data or projection data in this case, as shown in FIG. 19, the view number V1 enough to avoid degradation in spatial resolution may be set in the channel range of [C1, C2-1] which covers the set imaging view-field area. Further, the view numbers V3 may extremely be reduced in the channel ranges of [1, C1-1] and [C2, N] each corresponding to the area placed outside the set imaging view-field area, or the view numbers V3 may be set to V3=0.

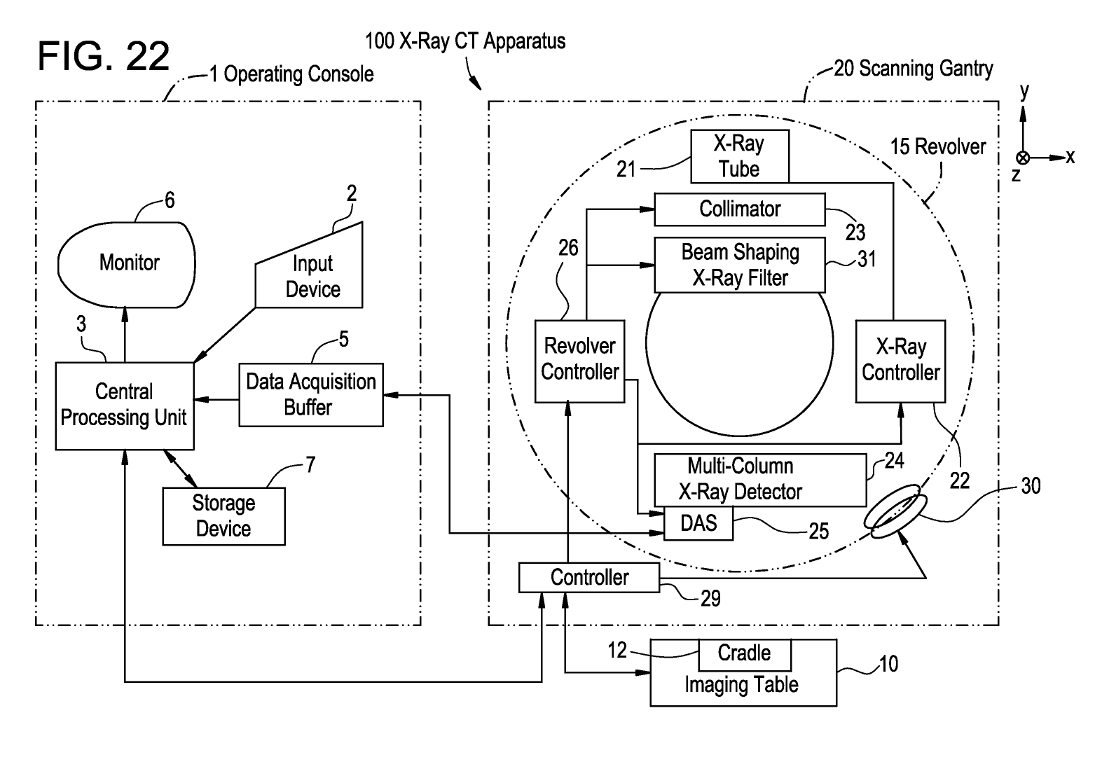

Incidentally, a system configuration diagram according to the sixth embodiment is given as shown in FIG. 22. The channel-direction collimator 31 is controlled by a rotating section controller 26 provided in a rotating section 15 of a scan gantry 20. The operation of each constituent element other than the channel-direction collimator 31 which controls the range of X rays applied in a channel direction in accordance with an imaging view-field area based on an imaging condition inputted from an input device 2, is similar to that illustrated in the first embodiment.

While there is a need to predict projection data for part of a subject unexposed to the X rays upon image reconstruction in this case and perform the image reconstruction, the details thereof have been described in the following patent.

### Seventh Embodiment

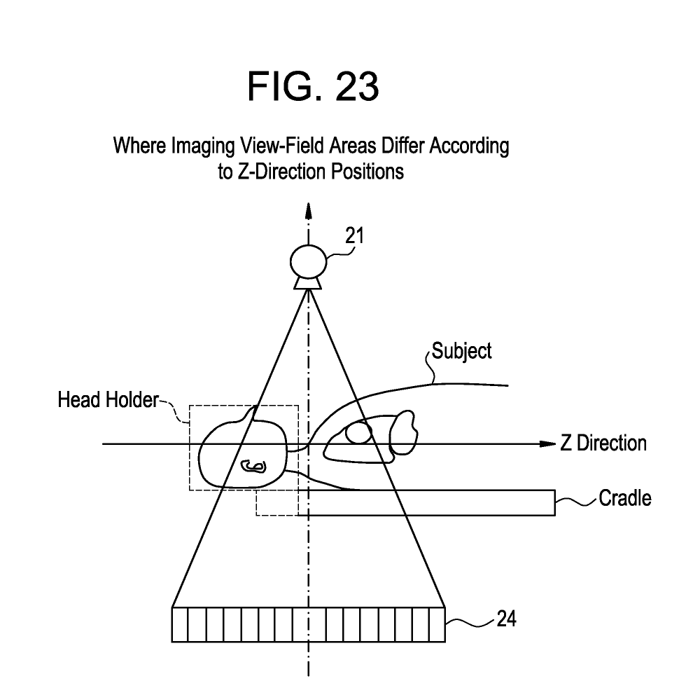

When the subject is imaged or photographed, e.g., the head, a neck region and shoulders are photographed as shown in FIG. 23, the section of the subject changes greatly and the optimum imaging view-field area also changes greatly.

If the neighborhood of the subject-existing area is set as the imaging view-field area as illustrated in the fourth embodiment, then the imaging view-field area changes depending upon z-direction coordinates. That is, the imaging view-field area changes for every row and the view numbers for the optimum respective channel positions also change, as shown in FIG. 23 in the case of a conventional scan (axial scan).

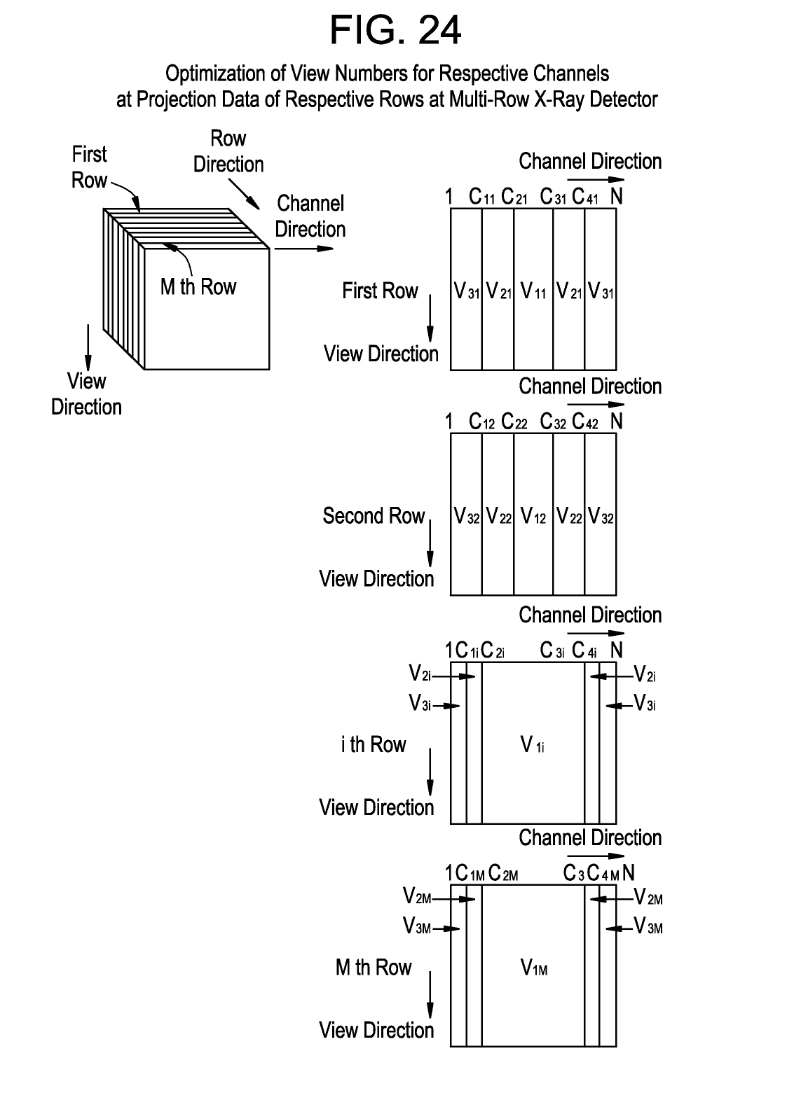

FIG. 24 shows optimization of view numbers for respective channels at X-ray detector data or projection data corresponding to respective rows of a multi-row X-ray detector at the execution of the conventional scan (axial scan). In FIG. 24, the view numbers are optimized as shown below at the respective channels of the multi-row X-ray detector corresponding to M rows.

In the case of X-ray detector data or projection data corresponding to the first row,

view number: V₃₁ in channel ranges [1, C₁₁-1] and [C₄₁, N]

view number: V₂₁ in channel ranges [C₁₁, C₂₁-1] and [C₃₁, C₄₁-1]

view number: V₁₁ in a channel range [C₂₁, C₃₁-1]

In the case of X-ray detector data or projection data corresponding to the second row,

view number: V₃₂ in channel ranges [1, C₁₂-1] and [C₄₂, N]

view number: V₂₂ in channel ranges [C₁₂, C₂₂-1] and [C₃₂, C₄₂-1]

view number: V₁₂ in a channel range [C₂₂, C₃₂-1]

In the case of X-ray detector data or projection data corresponding to the ith row,

view number: V₃₁ in channel ranges [1, C₁ᵢ-1] and [C₄ᵢ, N]

view number: V₂₁ in channel ranges [C₁ᵢ, C₂ᵢ-1] and [C₃ᵢ, C₄ᵢ-1]

view number: V₁ᵢ in a channel range [C₂ᵢ, C₃ᵢ-1]

In the case of X-ray detector data or projection data corresponding to the Mth row,

view number: V_{3M} in channel ranges [1, C_{1M}-1] and [C_{4M}, N]

view number: V_{2M} in channel ranges [C_{1M}, C_{2M}-1] and [C_{3M}, C_{4M}-1]

view number: V_{1M} in a channel range [C_{2M}, C_{3M}-1]

Image reconstruction in this case may make use of the image reconstructing method according to the first embodiment or the image reconstructing method according to the second embodiment.

When, however, an attempt is made to control the slice thickness in the z direction in the latter case, the view numbers set for every channel differ for every row. Therefore, the z filter cannot be convoluted in the row direction as in the z-filter convolution process at Step S4 of the first embodiment.

Assuming that it is desired to set a tomographic image G_{TH}(X, y, z) with a slice thickness d in a given z-direction position z₀ in this case, a z filter is convoluted, as viewed in the z direction, on a tomographic image corresponding to a slice thickness equivalent to one row of X-ray detector channels arranged in the z direction, of a two-dimensional X-ray area detector 24 of a matrix structure typified by a multi-row X-ray detector 24 or a flat panel X-ray detector, i.e., a tomographic image having a z-direction original slice thickness in CT or tomographic image space in which the image reconstruction has been finished, whereby a tomographic image whose slice thickness is thicker than the original slice thickness is image-reconstructed. z filters having weight coefficients (W₋ₙ, W₋ₙ₊₁, . . . W₋₁, W₀, W₁, . . . Wₙ₋₁, . . . , Wₙ) corresponding to a length of 2n+1 are convoluted on tomographic images G(x, y, z-n·Δz), G(x, y, z−(n−1)·Δz), . . . G(x, y, z−Δz), G(x, y, z), G(x, y, z+Δz), . . . G(x, y, z+(n−1)·Δz), . . . G(x, y, z+n·Δz) each having an original slice thickness Δd, which are image-reconstructed from respective rows determined by the conventional scan (axial scan) or cine scan. That is, the following equation is established.

$${G}_{TH} ( x , y , z ) = {\sum }_{i = - n}^{n} ( wi \cdot G ( x , y , z + i \cdot Δ z ) ) \qquad [ Equation 14 ]$$

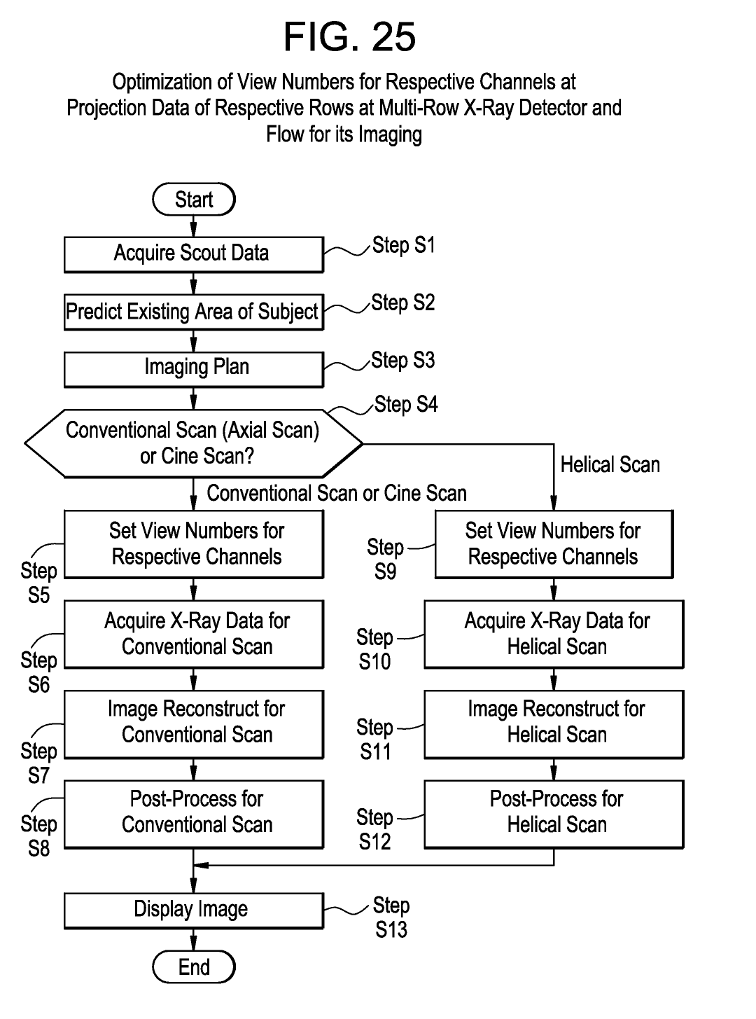

A flow for performing scans with these channel ranges and the values of view numbers being determined is as follows (refer to FIG. 25).

At Step S1, scout data acquisition is performed.

At Step S2, a subject-existing area is predicted.

At Step S3, an imaging or photographing plan or program is carried out.

At Step S4, it is determined whether the conventional scan (axial scan) or cine scan, or a helical scan should be performed. When the conventional scan (axial scan) or the cine scan is selected, the flow proceeds to Step S5. When the helical scan is selected, the flow proceeds to Step S9.

At Step S5, the view number for each channel is set.

At Step S6, conventional scan X-ray data acquisition is carried out.

At Step S7, conventional scan image reconstruction is executed.

At Step S8, a conventional scan post-process is executed.

At Step S9, the view number for each channel is set.

At Step S10, helical scan X-ray data acquisition is performed.

At Step S11, helical scan image reconstruction is performed.

At Step S12, a helical scan post-process is carried out.

At Step S 13, an image display is performed.

At Step S1, a subject is placed on its corresponding cradle 12 and thereafter a 0-degree direction scout image in an imaging or photographing range is scout-image photographed in a 90° direction.

At Step S2, the subject-existing area is predicted at each z-direction coordinate position approximately in ellipsoid form as a three-dimensional area from the 0-degree direction scout image and the 90° direction scout image as shown in FIG. 29.

At Step S3, imaging areas for respective portions or regions at the respective z-direction coordinate positions are optimally determined from the subject-existing areas at the respective z-direction positions determined at Step S2, whereby the imaging plan is made.

At Step S4, the flow proceeds to Step S5 if the conventional scan (axial scan) or the cine scan is taken, whereas if the helical scan is taken, then the flow proceeds to Step S6.

At Step S5, the view numbers for the respective channels corresponding to the respective rows at the respective z-direction coordinate positions are set from the imaging areas at the respective z-direction coordinate positions of the respective regions.

At Step S6, data acquisition for the conventional scan (axial scan) or the cine scan is performed in accordance with the view numbers for the respective channels at the respective z-direction coordinate positions set at Step S5.

At Step S7, the image reconstruction of the divided projection data shown in FIG. 11 is performed in accordance with the view numbers for the respective channels of the respective rows shown in FIG. 24.

Incidentally, the image reconstruction may be carried out by re-sampling the view numbers different for every channel position as shown in FIG. 10.

At Step S8, a process similar to the post-process employed in the first embodiment may be performed.

At Step S9, the view numbers for the respective channels of the rows at the respective z-direction coordinate positions are set by the imaging areas at the respective z-direction coordinate positions of the respective regions.

At Step S10, data acquisition for the helical scan is performed in accordance with the view numbers for the respective channels at the individual z-direction coordinate positions set at Step S9.

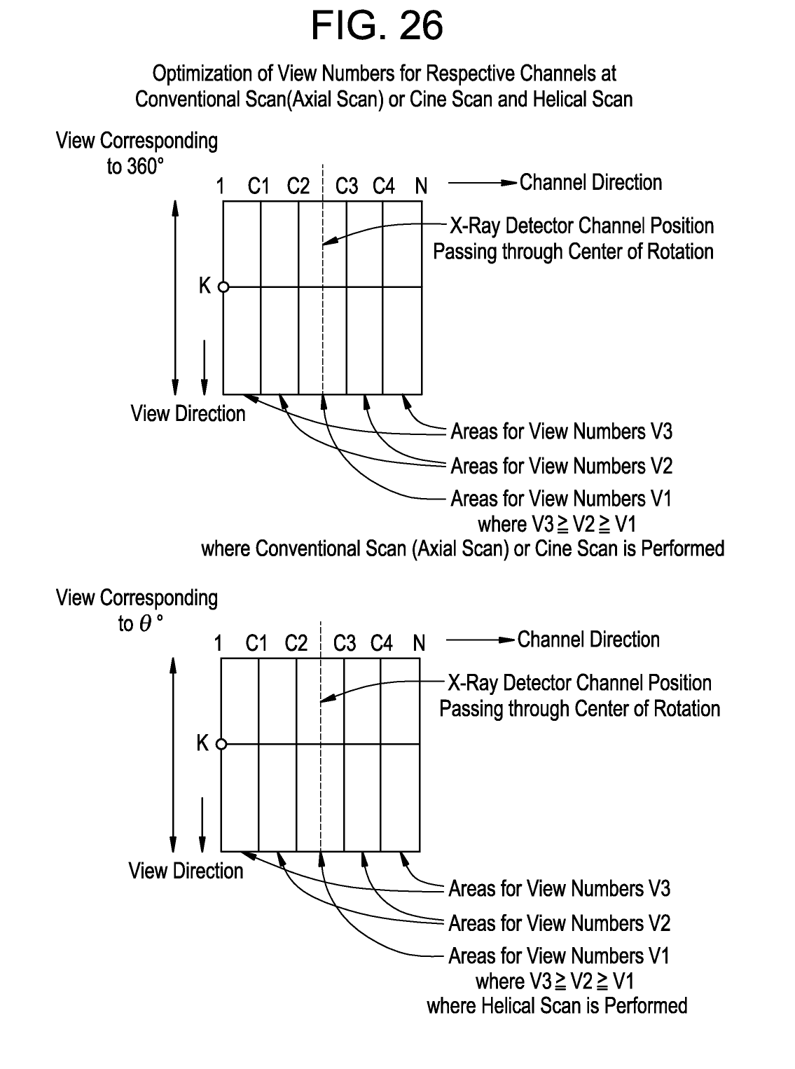

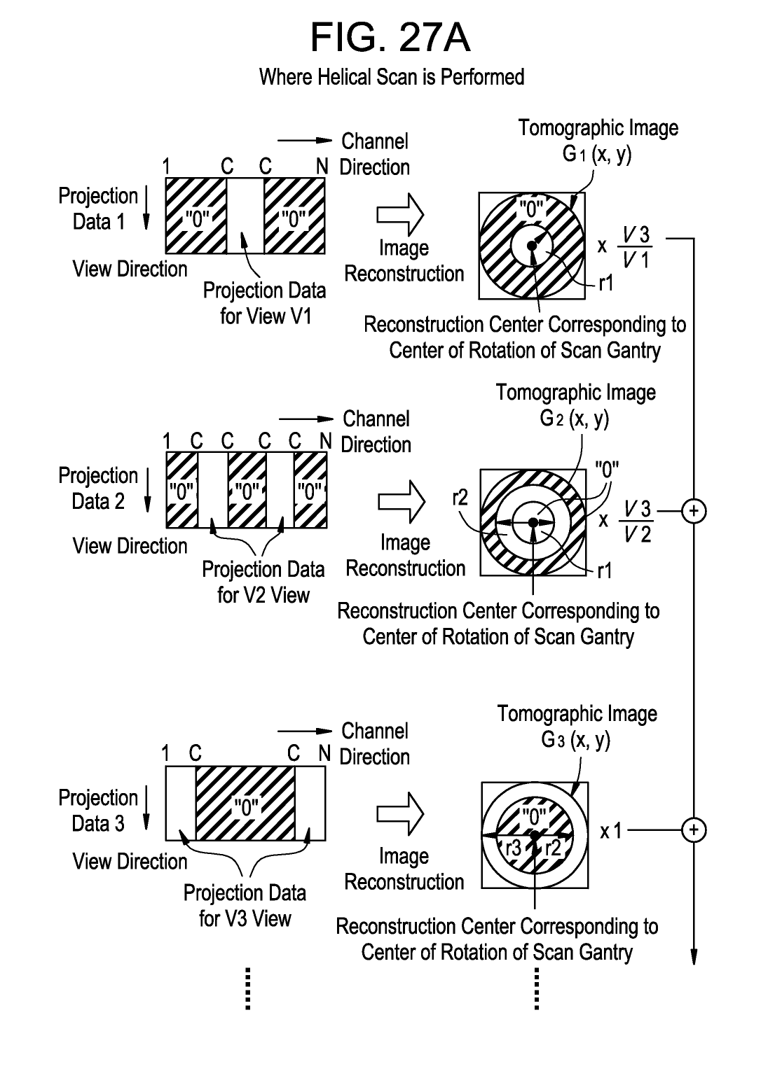

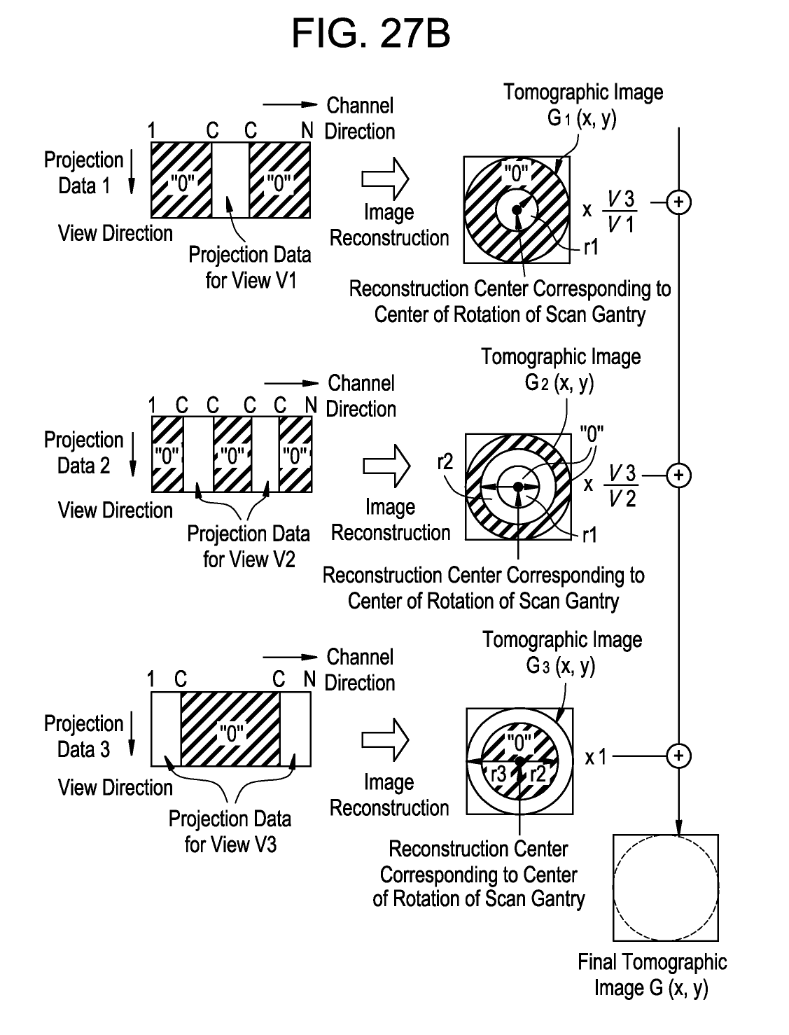

At Step S11, the projection data divided for every view range of each row is divided every channel range in accordance with view numbers for respective channels of respective rows shown in FIG. 26 thereby to perform image reconstruction (see FIG. 27).

At Step S12, a process similar to the post-process employed in the first embodiment may be executed.

At Step S13, an image-reconstructed CT or tomographic image is displayed in the form of an image.

According to the X-ray CT apparatus of the present invention or the X-ray CT imaging method, the above X-ray CT apparatus 100 brings about the effect of realizing an exposure reduction of the conventional scan (axial scan) or the cine scan, or the helical scan at an X-ray cone beam expanded in the z direction, which has been present at the start and end of the conventional scan (axial scan) or the cine scan or the helical scan of the X-ray CT apparatus having the two-dimensional area X-ray detector of the matrix structure typified by the conventional multi-row X-ray detector or the flat panel X-ray detector.

Incidentally, the image reconstructing method may adopt a three-dimensional image reconstructing method based on the Feldkamp method known to date. Further, another three-dimensional image reconstructing method may be adopted. Alternatively, a two-dimensional image reconstructing method may be adopted.

In the present embodiment, the row-direction (z-direction) filters different in coefficient for every row are convoluted, thereby adjusting variations in image quality and realizing a uniform slice thickness, artifacts and the image quality of noise at each row. Although various filter coefficients are considered therefore, any can bring about a similar effect.

Although the present embodiment has been described on the basis of the medical X-ray CT apparatus, it can be made available to an X-ray CT-PET apparatus utilized in combination with an industrial X-ray CT apparatus or another apparatus, an X-ray CT-SPECT apparatus utilized in combination therewith, etc.

In the present embodiment, the channel ranges are divided symmetrically or approximately symmetrically with the X-ray detector channel passing through the center of rotation as the center line as shown in FIG. 9. However, an actual multi-row X-ray detector is configured in module units such as 16 channels or 24 channels per module of an X-ray detector. Switching between view numbers in the module units is realistic. Therefore, the channel ranges are divided at the cut between the respective modules without making the above symmetry with the channel passing through the center of rotation being placed on the center line, and the view numbers may also be set to the respective channel ranges.

In the present embodiment, the view numbers for the X-ray data acquisition at the respective channels or channel ranges is preferably determined in proportion to the distance from the channel position of the X-ray detector passing through the center of rotation or the distance along the circular arc of the arcuate X-ray detector. However, it is realistically common that the data acquisition system (DAS) 25 controls the view numbers for every channel range in a given range with the number of channels corresponding to respective detector module units or units equivalent to plural times the detector module unit being defined as the unit. Therefore, the view numbers for the individual channel ranges may be controlled approximately in proportion to the distance from the center of rotation.

Although the present embodiment has shown the example in which the number of channel ranges is provided three and the type of view number is set to three, or the number of channel ranges is provided two and the type of view number is set to two, similar effects can be brought about even though these figures increase or decrease.

In the fifth embodiment, the subject-existing area has been predicted from the scout images in the 0° and 90° directions. However, the direction of each scout image is not limited to the z direction and may further be set to many directions or the like. Alternatively, a method of predicting a subject-existing area by an optical outer appearance image without predicting the subject-existing area by the X-ray based scout images.

1. An X-ray CT apparatus comprising:
X-ray data acquisition means for acquiring X-ray projection data transmitted through a subject lying between an X-ray generator and an X-ray detector detecting X-rays in opposition to the X-ray generator, while the X-ray generator and the X-ray detector are being rotated about the center of rotation lying therebetween;
image reconstructing means for image-reconstructing the projection data acquired from the X-ray data acquisition means;
image display means for displaying an image-reconstructed tomographic image; and
wherein said X-ray data acquisition means include means for performing X-ray data acquisition based on a plurality of types of X-ray data acquisition view numbers per rotation.

2. The X-ray CT apparatus according to claim 1, wherein said X-ray data acquisition means include means for performing X-ray data acquisition at a plurality of types of different X-ray data acquisition view numbers depending upon channel positions.

3. The X-ray CT apparatus according to claim 1, wherein said X-ray data acquisition means include means for acquiring X-ray data small in view number in channels located in the vicinity of the center of rotation and large in view number in channels at positions spaced away from an X-ray detector channel position passing through the center of rotation.

4. The X-ray CT apparatus according to claims 1, wherein said X-ray data acquisition means include means for performing X-ray data acquisition at a plurality of types of different X-ray data acquisition view numbers depending upon distances from an X-ray detector channel position passing through the center of rotation to respective channel positions.

5. The X-ray CT apparatus according to claims 1, wherein said X-ray data acquisition means include means for performing X-ray data acquisition at plural types of view numbers, based on X-ray data acquisition view numbers proportional to distances from an X-ray detector channel position passing through the center of rotation to respective channel positions, or about said X-ray data acquisition view numbers.

6. The X-ray CT apparatus according to claims 1, wherein said X-ray data acquisition means include means for performing X-ray data acquisition at view numbers different for every channel, depending upon reconstruction functions.

7. The X-ray CT apparatus according to claims 1, wherein said X-ray data acquisition means include means for performing X-ray data acquisition at view numbers different for every channel, depending upon the size of each imaging view field.

8. The X-ray CT apparatus according to claims 1, wherein said X-ray data acquisition means include means for performing X-ray data acquisition at view numbers different for every channel, depending upon z-direction coordinate positions.

9. The X-ray CT apparatus according to claims 1, wherein said X-ray data acquisition means include means for acquiring X-ray data by using a multi-row X-ray detector.

10. The X-ray CT apparatus according to claims 1, wherein said X-ray data acquisition means include mean for acquiring X-ray data using a two-dimensional X-ray area detector.

11. The X-ray CT apparatus according to claims 9, wherein said X-ray data acquisition means includes means for performing data acquisition at X-ray data acquisition view numbers different for every channel independently for every row.

12. The X-ray CT apparatus according to claims 10, wherein said X-ray data acquisition means includes means for performing data acquisition at X-ray data acquisition view numbers different for every channel independently for every row.
